SEQUENCE LISTING (7 sequences)

SEQ ID NO: 1 - protein, 43 aa, Artificial Sequence
  Description of Artificial Sequence Synthetic polypeptide
TGGPTIKPPKPPKPAPNLLGGPDKTHTKPPKPAPELLGGPGTG

SEQ ID NO: 2 - DNA, 18 bp, Artificial Sequence
  Description of Artificial Sequence Synthetic oligonucleotide
taccgcagccatcagagt

SEQ ID NO: 3 - protein, 6 aa, Artificial Sequence
  Description of Artificial Sequence Synthetic 6xHis tag
HHHHHH

SEQ ID NO: 4 - protein, 40 aa, Artificial Sequence
  Description of Artificial Sequence Synthetic polypeptide
GGGGSGGGGSGGGGSGGGGSGGGGSGGGGSGGGGSGGGGS

SEQ ID NO: 5 - protein, 11 aa, Human immunodeficiency virus
  Type 1
GATPQDLNTML

SEQ ID NO: 6 - protein, 10 aa, Artificial Sequence
  Description of Artificial Sequence Synthetic peptide
GGCGHHHHHH

SEQ ID NO: 7 - protein, 8 aa, Artificial Sequence
  Description of Artificial Sequence Synthetic peptide
GGHHHHHH

(19) United States
(12) Patent Application Publication
(10) Pub. No.: US 2013/0040845 A1
(43) Pub. Date: Feb. 14, 2013

(54) METHOD FOR SCREENING RECEPTORS/LIGANDS INTERACTIONS

(71) Applicants: Timothy A. Springer, Chestnut Hill, MA (US); Jongseong Kim, Brookline, MA (US)

(72) Inventors: Timothy A. Springer, Chestnut Hill, MA (US); Jongseong Kim, Brookline, MA (US)

(73) Assignee: IMMUNE DISEASE INSTITUTE, INC., Boston, MA (US)

(21) Appl. No.: 13/577,935

(22) PCT Filed: Feb. 14, 2011
(86) PCT No.: PCT/US2011/024690

§ 371 Date: Oct. 25, 2012

Related U.S. Application Data
(60) Provisional application No. 61/304,891, filed on Feb. 16, 2010.

(51) Int. Cl. G01N 33/53; C40B 30/04; G01N 21/17

(52) U.S. Cl. 506/9; 436/501

(57) ABSTRACT

Embodiments of the invention herein relate to methods of studying binding interactions between two entities and methods for screening of modulators of such binding interactions, in particular, the protein-protein interaction observed in receptor-ligand interactions.

### GOVERNMENT SUPPORT

This invention was made with Government support under contract No. HL 48675 awarded by the National Institutes of Health. The Government has rights in the invention.

### BACKGROUND OF INVENTION

Protein-protein interactions are an essential key in all biological processes, from replication and expression of genes to the morphogenesis of organisms. A multitude of biochemical, biophysical and theoretical methods are available for studying protein-protein interactions. Biochemical methods include protein co-immunoprecipitation, bimolecular Fluorescence Complementation (BiFC), affinity electrophoresis, label transfer, yeast two hybrid, tandem affinity purification (TAP), chemical crosslinking with/without MALDI mass spectrometry, SPINE (Strep-protein interaction experiment) followed by quantitative immunoprecipitation combined with knock-down (QUICK) relies on co-immunoprecipitation, quantitative mass spectrometry (SILAC) and RNA interference (RNAi). These methods detect interactions among endogenous non-tagged proteins. Biophysical methods include dual polarisation interferometry (DPI), static light scattering (SLS), dynamic light scattering (DLS), surface plasmon resonance, fluorescence correlation spectroscopy, fluorescence resonance energy transfer (FRET), and nuclear magnetic resonance.

While these methods have been successfully applied for protein studies, there are some drawbacks. In the yeast two hybrid method, the fusion proteins need to be translocated to the nucleus, which is not always evident. Proteins with intrinsic activation properties can cause false positives. Moreover, interactions that are dependent upon secondary modifications of the proteins, such as protein phosphorylation, cannot easily be detected. Some methods are dependent on the availability of suitable antibodies. Co-immunoprecipitation experiments reveal direct and indirect interactions. Weaker interactions can be missed by co-immunoprecipitation experiments as the weaker interaction dissociates during the precipitation step. Where the protein-protein interactions take place in vivo, the method does not easily allow the study of single interaction, the effects of small molecules on the interaction or the identification of compounds that can modulate a specific protein-protein interaction. Only small molecules and compounds that can enter a cell and has no detrimental effects on the general function of a cell will permit any such studies. Similarly such method does not permit binding kinetic studies of two interacting proteins.

### SUMMARY OF THE INVENTION

Embodiments described herein relate to methods of studying binding interactions between two entities and methods for screening of modulators of protein-protein interactions, in particular, receptor-ligand interactions. The dissociation and association of two proteins or binding portions thereof, and the related dissociation and association rate constants can be studied by the methods.

The inventors have found that by using a single chimeric fusion polypeptide that comprise a receptor portion and a ligand portion within a single polypeptide, wherein the receptor portion and the ligand portion are in close proximity, binding between the receptor portion and ligand portion occurs. Using an optical tweezer to pull the two ends of such a chimeric polypeptide in opposite directions, thereby extending or stretching out the chimeric polypeptide, strain is applied to the interaction between the receptor and its ligand in the chimeric polypeptide. The inventors were able to study the minute external forces needed to stretch out the fusion polypeptide such that the interaction between the receptor portion and ligand portion is overcome by the external force applied. In addition, the inventors were able to study the effects of receptor agonist/activator or antagonist/inhibitor on the external forces needed to disrupt the interaction between the receptor portion and ligand portion in the chimeric polypeptide. The inventors found that this technique allows for the screening of molecules that can modulate the interaction between a receptor portion and a ligand portion. This technique can be used for identifying agonist/activator or antagonist/inhibitor on the basis of an increase or decrease in the external force needed to disrupt the interaction between the receptor portion and ligand portion in the presence of agonist/activator or antagonist/inhibitor compared to in the absence of the agonist/activator or antagonist/inhibitor. Other parameters useful for identifying agonist/activator or antagonist/inhibitor includes but are not limited to the changes in the dissociation and association rate constants for the interaction between a receptor portion and a ligand portion. In essence, the inventors have developed an assay for single molecule measurements of reversible receptor-ligand bond interactions. This assay is also applicable for the interaction between a receptor portion and a ligand portion wherein the receptor portion and the ligand portion are in separate polypeptides and not on a single chimeric polypeptide.

Accordingly, provided herein is a method of screening for a modulator of an interaction between a receptor and a ligand pair, the method comprising: (a) contacting a ligand-bound-receptor protein with an agent; (b) extending the ligand-bound-receptor protein; (c) monitoring a signal that represents the protein existing in either a ligand-bound state or in a ligand-unbound state and the transition between the two states; and (d) comparing the signal with a reference signal, wherein a deviation from a reference indicate that the agent is a modulator.

In one embodiment, the reference is that of the ligand-bound-receptor protein in the absence of a modulator.

In one embodiment, the extending of the ligand-bound-receptor protein occurs with an optical tweezer or an atomic force microscope (AFM).

In one embodiment, the ligand-bound-receptor protein is extended to achieve a constant tension force between the ends of the protein. In this embodiment, the signal is the length of the extended protein maintained over time under the constant tension force. In another embodiment, the ligand-bound-receptor protein is extended to disrupt the ligand/receptor interaction therein the ligand-bound-receptor protein. In this embodiment, the signal is one that indicates the force at which the ligand/receptor interaction dissociation occurs.

In one embodiment, the extending of the ligand-bound-receptor protein occurs with a mobile focus laser light, a cantilever, or a positioner in the AFM.

In one embodiment, the signal is a force required to dissociate or disrupt the ligand receptor interaction and/or produce an increase in extension of the ligand-bound-receptor protein.

In one embodiment, the signal is a rate of dissociation of the ligand receptor interaction and/or a dissociation constant of the rate. Such rates are computed from data related to force required to dissociate the ligand receptor interaction and/or produce an increase in extension of the ligand-bound-receptor protein.

In one embodiment, the ligand-bound-receptor protein is extended to achieve a constant tension force between the ends of the protein. In such an embodiment, the signal is an extension or a displacement of the ends of the protein (i.e., the length of the protein in the instrument set up) required for maintaining the constant tension force between the ends of the protein.

In one embodiment, a positive deviation of at least 10% from the reference indicates that the modulator is an agonist/activator of the receptor-ligand interaction.

In one embodiment, a negative deviation of at least 10% from the reference indicates that the modulator is an antagonist/inhibitor of the receptor-ligand interaction.

In one embodiment, the ligand-bound-receptor protein is a fusion chimeric protein comprising (1) a receptor or ligand-binding fragments thereof and (2) a ligand or receptor-binding fragment thereof, wherein the receptor or ligand-binding fragments thereof and the ligand or receptor-binding fragment thereof are fused together in a single polypeptide;

In one embodiment, the ligand-bound-receptor protein is a complex of two independent polypeptides, a first polypeptide and a second polypeptide, wherein the first polypeptide comprises a receptor or ligand-binding fragments thereof and the second polypeptide comprises a ligand or receptor-binding fragment thereof; wherein the complexing is by way of the ligand-receptor interaction (see FIGS. 10A, 11A and 12A); and wherein the two polypeptides are linked by non-covalent bonds located at the ligand binding/receptor-binding regions of the polypeptides. In one embodiment, the two independent polypeptides are linked to each other by non-covalent bonds located at the non-ligand binding/non-receptor-binding regions of the polypeptides. In one embodiment, the two independent polypeptides are linked to each other by covalent bonds located at the non-ligand binding/non-receptor-binding regions of the polypeptides, e.g., disulfide bridges.

As used herein, “non-ligand binding/non-receptor-binding regions” refer to the parts of a polypeptide described herein that are not involved in the receptor/ligand interaction.

In one embodiment, the ligand is a natural ligand of the receptor.

In one embodiment, the ligand is an artificial ligand of the receptor such as a synthetic drug or viral/pathogen component. The ligand is no one that the receptor normally binds to in nature. For example, the artificial ligand is a protein from a pathogen, or a Fab fragment of antibody that binds the receptor protein, e.g., from a chimeric and humanized monoclonal antibodies raised against the receptor protein.

In one embodiment, in the context of a chimeric fusion polypeptide having a receptor portion and a ligand portion, the receptor or ligand-binding fragments thereof and the ligand or receptor-binding fragment thereof are separated by a linker molecule. In one embodiment, the receptor or ligand-binding fragments thereof and the ligand or receptor-binding fragment thereof are separated by a spacer linker peptide. In one embodiment, the spacer linker peptide has at least one amino acid residue and up to 200 amino acid residues.

In one embodiment, the ligand-bound receptor protein is tethered to at least a solid surface, e.g., via handles. Preferably, the tethering occurs at the ends of the ligand-bound receptor. More preferably, the end of the ligand-bound receptor is distal to the receptor/ligand interaction portions, such that the tethering does not interfere with the receptor/ligand interaction that is being studied. In one embodiment, both amino and carboxyl ends of the protein are tethered to at least a solid surface via a handle for use with an optical tweezer or an atomic force microscope (AFM), wherein the independent ends of the protein are each tethered to a different solid surface. In one embodiment, only one end of the protein is tethered to a solid surface via a handle for use with an optical tweezers or an AFM.

In one embodiment, the handle is a double-stranded DNA (dsDNA). In one embodiment, the dsDNA handle comprises thiol-derivatized bases.

In one embodiment, the receptor-ligand pair is VWF A1 domain and GP1bα subunit.

In other embodiments, the receptor-ligand pair is selected from the group consisting of α4b7 integrin-madcam-1, αL integrin I domain—ICAM-1(D1+D2), αL integrin I domain—ICAM-3 (D1); and fimH pilin+lectin domain—N-linked carbohydrate.

### BRIEF DESCRIPTION OF THE DRAWINGS

FIGS. 1A-1E show the model for mechanoenzymatic cleavage of unusually large von Willebrand factor (ULVWF) in circulation.

FIG. 1A shows the shear flow in a vessel and elongational flow at a site of bleeding.

FIG. 1B shows the shear flow that is represented as elongational flow superimposed on rotational flow.

FIG. 1C illustrates VWF molecules in an elongating, compressing, or tumbling configuration during shear flow.

FIG. 1D shows the peak force as a function of VWF monomer position in a VWF multimer chain of 200, 100, or 50 monomers at 100 dyn/cm². Dashed line shows the most likely unfolding force for the A2 domain at a loading rate of 25 pN/sec.

FIG. 1E shows the schematic of a VWF monomer, with the N-terminal end as a triangle symbol, the A2 domain as a spring symbol, and the C-terminal end as a circle symbol. Elongation results in unfolding of some A2 domains, some of which are cleaved (arrows). The resulting fragments are shown.

FIG. 2 illustrates the players involved in and the events leading up to a VWF-mediated platelet aggregation. Mannucci, N Engl J Med, 2004.

FIG. 3A shows the experimental setup for the measuring the interaction of the VWF A1 domain with the platelet glycoprotein Ibα (GAP1bα) subunit using optical tweezers. The interaction comprises binding and unbinding between to A1 and GAP1bα. Covalently tethered A1-GP1bα is coupled to double-stranded DNA handles that are attached at their other ends to beads held by an adjustable optical trap and a fixed micropipette.

FIG. 3B shows a representative force-extension trace of a protein A1-GP1bα chimeric fusion polypeptide during repeated force increase and decrease at constant pulling and relaxation rates. The A1-GP1bα chimeric fusion polypeptide is constructed as a single polypeptide comprising the A1 domain (i. e. the receptor) and the GP1bα subunit (i. e. the ligand of the A1 domain).

FIG. 3C shows the force-extension data and force clamp data of A1-GP1bα unbinding fitted to the Worm Like Chain (WLC) model used to describe polymer stretching. Solid squares represent rip data for disrupting the interaction between A1-GP1bα. Open triangles represent force clamp data.

FIG. 4A shows the schematic design of an embodiment of a VWF A1-GP1bα chimeric fusion polypeptide constructed for covalently tethering additional molecules to the termini of the polypeptide and an embodiment of a corresponding VWF A1-GP1bα chimeric polypeptide serving as a control. The control VWF A1-GPIbα chimeric polypeptide is designed to have non-interacting A1-GP1bα portions in the chimeric polypeptide. Peptide ‘GGCG—H(6)’ is disclosed as SEQ ID NO: 6 and peptide ‘GG—H(6)’ is disclosed as SEQ ID NO: 7.

FIG. 4B shows the protein purification of the VWF A1-GP1bα chimeric fusion protein using Ni-NTA affinity and size exclusion columns.

FIG. 4C is a 4-20% native gel that is stained with SYBR® Green nucleic acid gel stain showing the antibody shift assay that indicates that the chimeric fusion porlypeptide is coupled to DNA handles.

FIGS. 5A-5C are histograms showing the unbinding force distribution of the A1-GP1bα interaction at different pulling rates.

FIGS. 5D-5F are histograms showing the unbinding forces distributions of the A1-GP1bα interaction in presence or absence of agonists/activators at a fixed pulling rate.

FIG. 6A is a graph showing the interaction bond lifetimes τ(F) at constant force estimated from the distribution of unbinding forces collected in the experiments exemplified in FIG. 5. Data are estimated from pulling experiments at 5 nm/sec (filled squares), 10 nm/sec (filled circles), 20 nm/sec (open triangles), 40 nm/sec (open squares). The filled circle shows the lifetime measured by Sadler et al. in bulk phase measurements of ¹²⁵I-labeled A1 domains to GP1bα-agarose beads and to platelets. Enlarged inset shows additional bond lifetime measurements (gray filled diamonds) at constant force in force clamp experiments.

FIG. 6B is a graph showing the interaction bond lifetimes τ(F) at constant forces in the presence of ristocetin (filled circles) and botrocetin (open squares). Data are estimated from the distribution of unbinding forces collected in the experiments exemplified in FIG. 5E and F. The constant K_{off}^{o} are extrapolated by occurrence-weighted least squares fit to k_{off}=K_{off}⁰exp(σF/k_{B}T).

FIGS. 7A-7D show representative extension versus time traces for the A1-GP1bα interactions at various constant forces in force clamping experiments. The increase in frequency of an extension of ˜10 nm represents the characteristic unbinding rate of A1-GP1bα intrecation at the particular constant force applied.

FIGS. 7E and 7F show the two distinct bond dissociation rates the A1-GP1bα interaction in the VWF A1-GP1bα chimeric fusion polypeptide in force clamping experiments conducted at constant forces of 10.05 pN (FIG. 7E) and 10.27 pN (FIG. 7F).

FIG. 8 shows the schematic model for the A1-GP1bα interaction. Two dissociation pathways (A to C and B to C) are seen in distinct force regimes.

FIG. 9A shows an example of a DNA with a thiol tethered nucleoside base and the chemical reaction leading to the formation of a disulfide bridge with a cysteine residue in a protein.

FIG. 9B is a schematic diagram showing the steps in crosslinking double stranded DNA (dsDNA) to a protein.

FIG. 10A is a schematic diagram showing the complexing of two separate proteins (103 and 107) via the interaction of the receptor-binding portion 105 of a ligand protein 107 with the ligand-binding portion 101 of a receptor protein 103 in a set up for optical tweezer manipulation.

FIG. 10B is a schematic diagram showing the interaction of the receptor-binding portion 105 with the ligand-binding portion 101 of a single chimeric fusion protein 117 in a set up for optical tweezer manipulation. The chimeric fusion polypeptide comprises the receptor-binding portion 105 of a ligand protein and the ligand-binding portion 101 of a receptor protein in a single polypeptide.

FIG. 11A is a schematic diagram showing the complexing of two separate proteins (103 and 107) via the interaction of the receptor-binding portion 105 of a ligand protein 107 with the ligand-binding portion 101 of a receptor protein 103 in a set up for atomic force microscopy.

FIG. 11B is a schematic diagram showing the interaction of the receptor-binding portion 105 with the ligand-binding portion 101 of a single chimeric fusion protein 117 in a set up for atomic force microscopy. The chimeric fusion polypeptide comprises the receptor-binding portion 105 of a ligand protein and the ligand-binding portion 101 of a receptor protein in a single polypeptide.

FIG. 12A is a schematic diagram showing the complexing of two separate proteins (103 and 107) via the interaction of the receptor-binding portion 105 of a ligand protein 107 with the ligand-binding portion 101 of a receptor protein 103.

FIG. 12B is a schematic diagram showing the interaction of the receptor-binding portion 105 with the ligand-binding portion 101 of a single chimeric fusion protein 117 comprising the receptor and the ligand as a chimeric fusion polypeptide. The chimeric fusion polypeptide comprises the receptor-binding portion 105 of a ligand protein and the ligand-binding portion 101 of a receptor protein in a single polypeptide.

FIG. 13 shows a schematic diagram of the two modules in the construction of the integrin α4β7-MAdCAM-1 receptor-ligand pair.

FIG. 14A shows the histograms of the unbinding force distribution of the Pselectin-PSGL1 interaction collected at different pulling rates.

FIG. 14B shows the histograms of the unbinding force distribution of the A1-GP1bα interaction collected at different pulling rates.

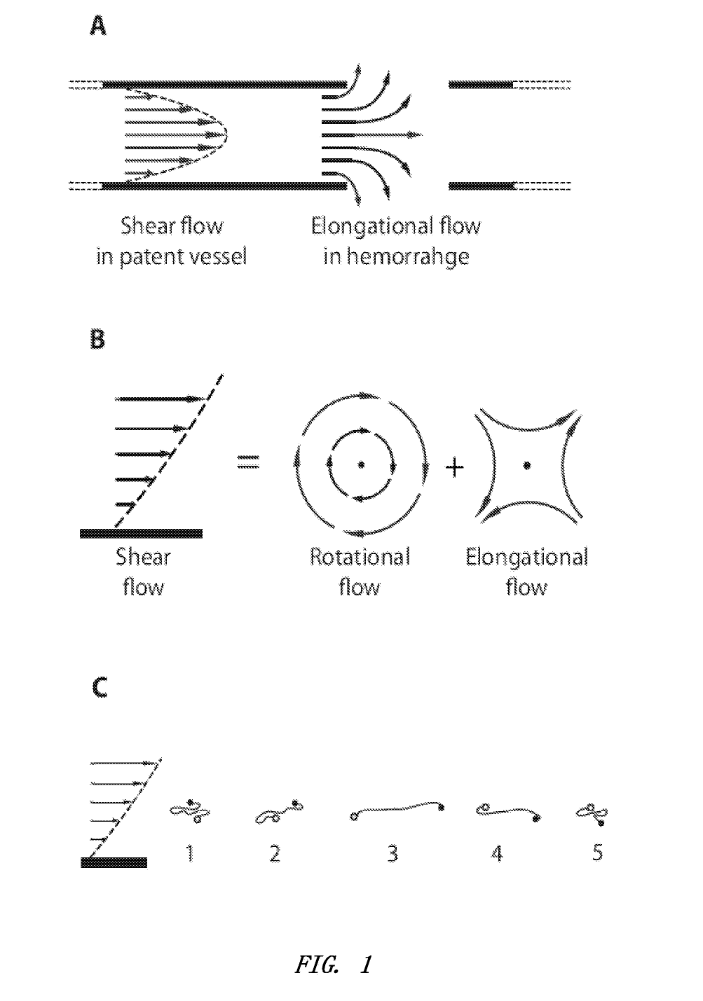

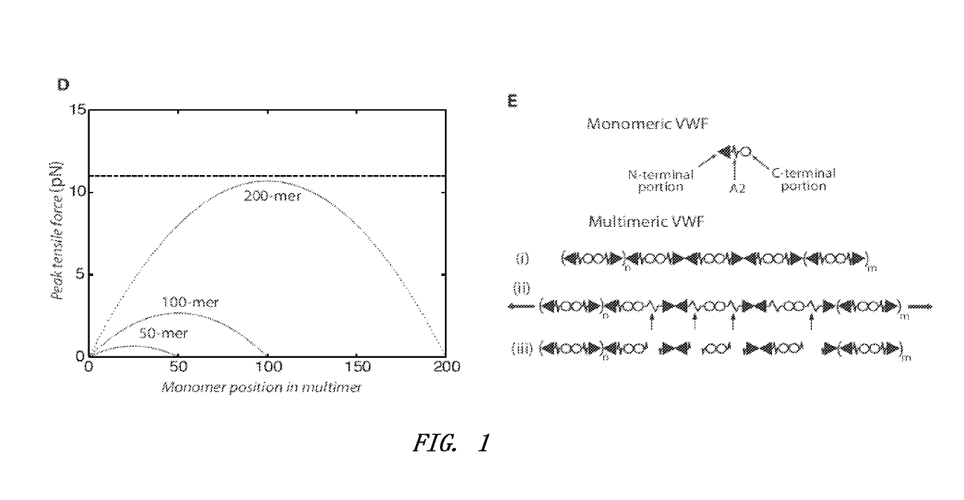

### DETAILED DESCRIPTION OF THE INVENTION

Embodiments described herein relate to the studying binding interactions between two entities and uses of such studies. Particularly, the embodiments relate to the methods of studying binding interactions between two entities and methods for screening of modulators of such binding interactions, e.g., protein-protein interactions, such as those of receptor-ligand interactions. The association and dissociation between two interacting proteins or binding portions thereof, and the related dissociation and association rate constants can be studied by the methods. The constants are useful indicators of changes affecting the interaction.

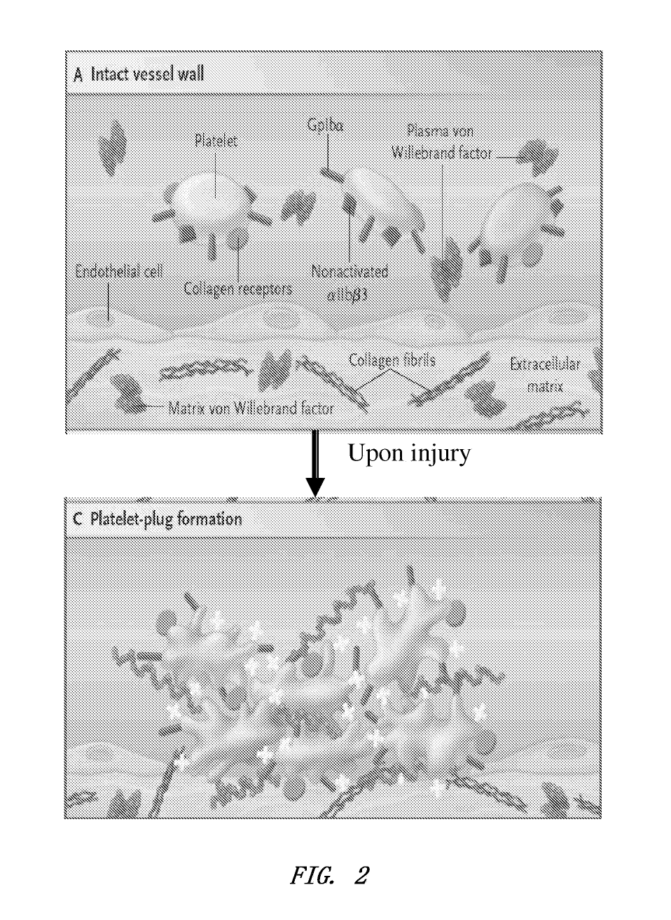

As proof-of-principle, in the example described herein, the inventors studied the interaction of the Von Willebrand factor (VWF) with platelet glycoprotein Ibα (GPIbα). VWF is a large multimeric protein with specific binding domains for specific binding partners (see FIG. 2). In the example, the VWF A1 domain is the binding domain for the GPIbα. Accordingly, with respect to the VWF A1 domain-GPIbα interaction being investigated by the inventors, VWF corresponded to a “receptor” and GPIbα corresponded to a “ligand” of VWF. Therefore, the VWF A1 domain is a ligand-binding fragment of the VWF receptor for the ligand GPIbα.

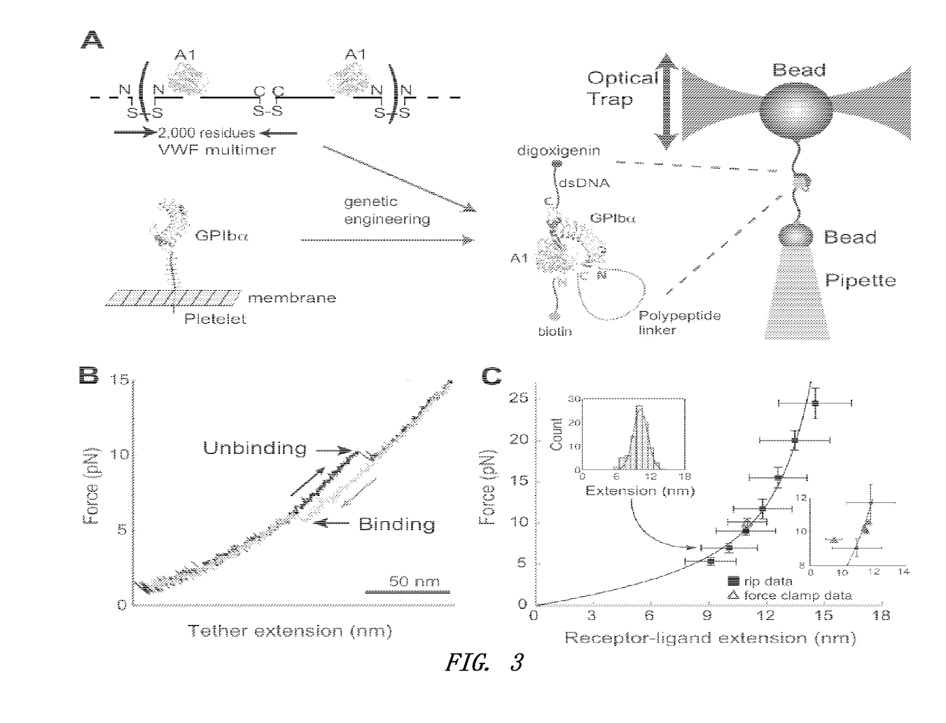

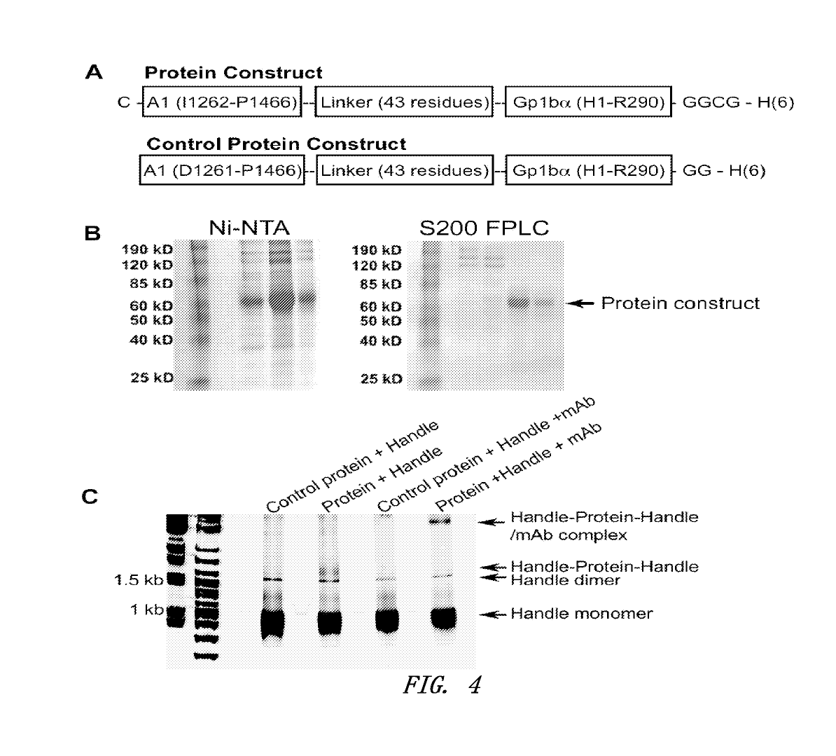

Using cell, molecular and genetic engineering methods known in the art, the inventors constructed a single polypeptide that comprises a VWF A1 domain and a GPIbα. The VWF A1 domain and GPIbα are arranged in tandem in the single polypeptide but are separated by a spacer linker peptide (see FIG. 3A and FIG. 4A). The close proximity of the VWF A1 domain and GPIbα within a single polypeptide facilitates their binding each other in their normal A1 domain-GPIbα interaction. In addition, when the VWF A1 domain/GPIbα interaction dissociates, their close proximity as a result of being linked together in a single polypeptide further facilitate re-binding again when conditions permit, e.g., when the dissociation/extension force is reduced to below that required for overcoming the interaction.

In the example described herein, the inventors tethered this single polypeptide to the solid surfaces of two different microspheres using double-stranded DNA (dsDNA) handles. One end of the chimeric polypeptide was tethered to a first dsDNA handle and the other end of the polypeptide was tethered to a second dsDNA handle. The polypeptide was coupled to the dsDNA handles via S—S-bond or disulfide bridges. The free ends of the dsDNA were then linked to digoxigenin or biotin (see FIGS. 3A, 4B & 4C, 10). The dsDNA coupled A1/GPIbα polypeptide was then mixed with beads that were coated with either anti-digoxigenin or streptavidin. The inventors then manipulated the beads with a pipette and a focus laser beam from an optical tweezer to stretch out or extend the A1/GPIbα polypeptide in order to study the minute forces, in pico Newtons (pN), required to dissociate the VWF A1 domain/GPIbα interaction. These are the force extension or force binding dissociation experiments. In addition, the inventors measured the extensions of the VWF A1 domain/GPIbα polypeptide that are required to keep the extension force constant. These are conducted using force clamp experiments.

While not wishing to be bound by theory, the intermolecular binding between a receptor and its ligand or between two proteins is held together by non-covalent bonds such as hydrogen bonds, hydrophobic interactions and/or van der Waals interactions. Similarly, non-covalent bonds such as hydrogen bonds, hydrophobic interactions and/or van der Waals interactions contribute to the intramolecular binding between two binding portions within a molecule, e.g., a polypeptide. Dissociating such bindings means breaking or overcoming the non-covalent bonds holding the receptor and its ligand or between two proteins binding partners together in the interaction. In the absence of an extraneous modulator, each interaction has a unique binding force. To dissociate the interaction, this unique force must be surpassed by an external force greater than that of the unique binding force. However in the presence of a modulator, the modulator can strengthen/enhance (i.e., an agonist/activator) or weaken/inhibits (i.e., an antagonist/inhibitor) the receptor-ligand or protein-protein interaction. In the former situation, wherein the modulator is a strengthener of the interaction, the presence of the modulator leads to an increase the force needed to dissociate the interaction when compared to the force needed in the absence of the modulator. For the latter situation, wherein the modulator weakens the interaction, the presence of the modulator leads to a decrease the force needed to dissociate the interaction when compared to the force needed in the absence of the modulator.

The effects of an agonist or an antagonist on an interaction studied in force clamp experiments are slightly different. In force clamp experiments, the bound protein is held at a particular constant tension by the extension force and the bound molecules take on a particular extension length and/or a specific range of extensions in order to maintain the particular constant tension within the extended bound protein. The extended protein held at a constant tension force oscillates between a bound form (which has a distinct extension length) and an unbound form (which has a distinct longer extension length). The particular extension or specific range of extensions, and the rate of oscillation between the bound form and unbound form for a protein held at a set constant tension is unique for each interaction between two proteins binding partners. For constant force-clamp experiments, the dissociative/unbinding rate is lower in the presence of a modulator that is a strengthener of an interaction compared to in the absence of the modulator. In the latter situation, the dissociative/unbinding rate is greater in the presence of such a modulator that is a weakener compared to in the absence of the modulator.

Accordingly, embodiments described herein provide a method for screening for a modulator of an interaction between a receptor and a ligand pair, the method comprising: (a) contacting a ligand-bound-receptor protein with an agent; (b) extending the ligand-bound-receptor protein; (c) monitoring a signal that represents the protein existing in either a ligand-bound state, in a ligand-unbound state or in a transition between the two states; (d) comparing the signal with a reference signal wherein a deviation from the reference indicate that the agent is a modulator.

In one embodiment, provided herein is a method of studying binding kinetics between at least two entities, the method comprising (a) contacting the entities together for binding to occur, forming a bound pair, (b) extending the bound pair, (c) monitoring a signal that represents the bound pair existing in either a bound state, in an unbound state or in a transition between the two states; and (d) computing the data obtained from step (c). In one embodiment, the entities are proteins with portions or functional fragments that bind each other. In one embodiment, the proteins are separate and independent polypeptide. In another embodiment, the entities are binding portions found within a single molecule. e.g., a single polypeptide. For example, the entities are the ligand-binding portion of a receptor protein and the receptor-binding portion of a ligand, both of which are found in a single polypeptide, as exemplified in the VWF A1 domain/GPIbα chimeric fusion polypeptide described herein. In one embodiment, the method further comprises contacting the bound pair with an agent, wherein the agent is a modulator of the interaction of the bound pair.

In one embodiment, the contacting involves applying the agent directly to the ligand-bound-receptor protein. In another embodiment, the contacting involves applying a solution comprising the agent to the ligand-bound-receptor protein, e.g., bathing the protein in a solution comprising the agent.

In one embodiment, the ligand-receptor interaction is established before the ligand-bound-receptor protein is tethered to at least one solid surface. That is, the ligand molecule/protein in allowed to interact and bind to the receptor protein, for example, in solution to form the ligand-bound-receptor protein before attaching thus formed ligand-bound-receptor protein to at least one solid surface. In one embodiment, the ligand-receptor interaction is established in the presence of the agent before the ligand-bound-receptor protein is tethered to at least one solid surface. That is, the ligand molecule in allowed to interact and bind to the receptor protein presence of the agent, for example, in solution to form the ligand-bound-receptor protein.

In another embodiment, the ligand-receptor interaction is established after the ligand-bound-receptor protein is tethered to at least one solid surface. That is, after the ligand molecule or protein is attached to at least one solid surface, and the receptor protein is also attached to at least one solid surface, the ligand molecule/protein and the receptor proteins are brought in very clone proximity for the ligand to interact and bind the receptor. In one preferred embodiment, the solid surface upon which the ligand molecule/protein is attached is not the same one as that upon which the receptor protein is attached. In one embodiment, the ligand-receptor interaction is established in the presence of the agent after the ligand-bound-receptor protein is tethered to at least one solid surface. That is, the ligand molecule in allowed to interact and bind to the receptor protein presence of the agent when the ligand molecule/protein and the receptor protein are brought in close proximity for binding to occur.

In one embodiment, the reference signal is that of the ligand-bound-receptor protein in the absence of a modulator.

In one embodiment, the ligand-bound-receptor protein or the bound pair is extended with an increasing external force that results in the disruption of the interaction within the protein or the bound pair. For example, in a force rupture experiment.

In another embodiment, the ligand-bound-receptor protein or the bound pair is extended with an external force that is kept constant. For example, in a force clamp experiment that is known in the art and as described herein.

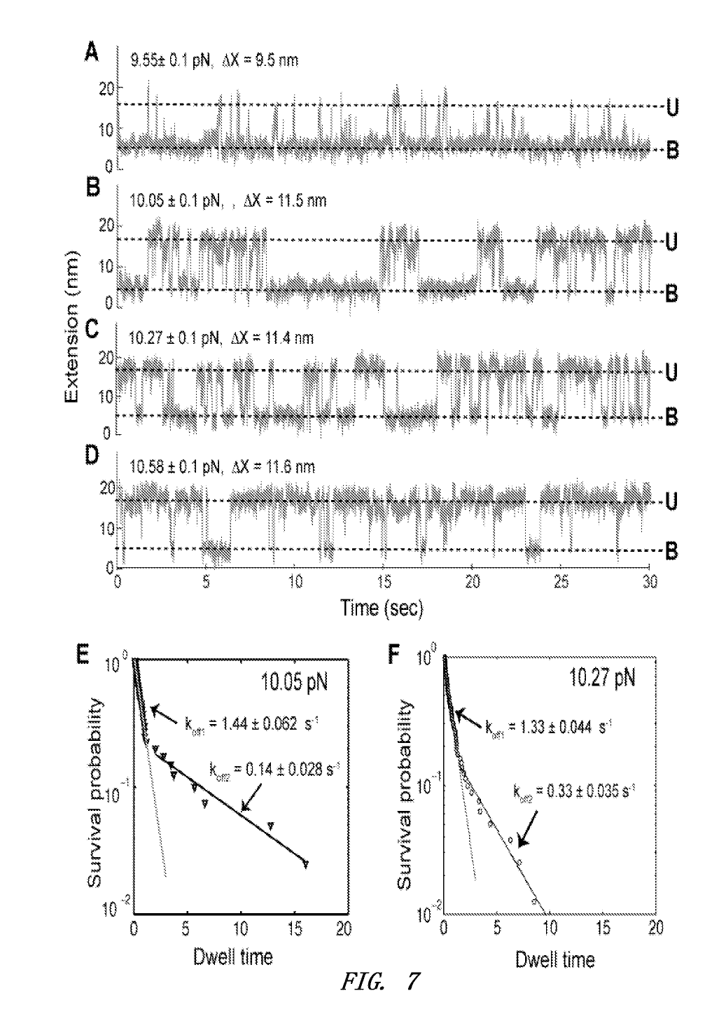

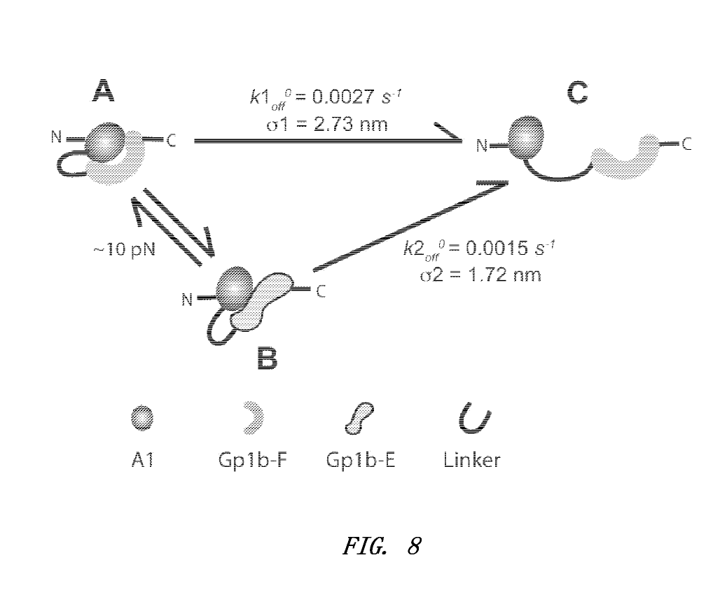

In one embodiment, the length of the extended protein is monitored over time when the external force that extends the protein is kept constant, e.g., in a force clamp experiment. See FIG. 7A-7D. In one embodiment, the frequency of the extended protein oscillating between the bound form and the unbound form is computed for each constant external force investigated.

In one embodiment, the extending of the ligand-bound-receptor protein occurs with an optical tweezer. As the name suggests, optical tweezers are means to manipulate small objects with light, including microscopic objects as small as a single atom. The radiation pressure from a focused laser beam traps small particles. This technique has been applied to a variety of biological systems, e.g., viruses, bacteria, living cells, organelles, small metal particles, and even strands of DNA. Applications include confinement and organization (e.g., for cell sorting), tracking of movement (e.g., of bacteria), application and measurement of small forces, and altering of larger structures (such as cell membranes). In the biological sciences, these instruments have been used to apply forces in the pN range, manipulate the positions of particles and to measure displacements in the nanometer (nm) range of objects ranging in size from 10 nm to over 100 mm.

For a typical optical tweezer, a laser beam is focused by a high-quality microscope objective to a spot in the specimen plane. This spot creates an “optical trap” which is able to hold a small particle at its center. The forces felt by this particle consist of the light scattering and gradient forces due to the interaction of the particle with the light. The basic physical principle underlying optical tweezers is the radiation pressure exerted by light when colliding with matter. For macroscopic objects, the radiation pressure exerted by typical light sources is orders of magnitude too small to have any measurable effects. Neglecting absorption, the forces exerted on the particle are caused by refraction and reflection of light. Typically, these forces are split into the gradient force that is directed in the direction of the light gradient (i.e. the laser focus) and the scattering force that is directed along the optical axis and pushes the particle out of the focus. Tightly focused laser beam produced, for example, by a microscope objective lens, can be used to trap small objects in three dimensions.

Optical tweezers have been used extensively not only to manipulate biomolecules and cells, but also to directly and accurately measure the minute forces (on the order of fractions of picoNewtons (pN)) involved. Most often, the biomolecules of interest are not trapped directly, but manipulated through functionalized microspheres. Methods of manipulating molecules with optics are well known in the art, for example, Mathias Salomo et al., 2008, Eur. Biophy. J., 37(6); Y. Chen, et al., 2009, Biophysical J., 96(3): 343a; M. Manosas, et al., 2007, Biophysical J., 92(9):3010-3021; U.S. Pat. Nos. 7,087,894; 7,248,413; 7,588,672; 7,612,355; and U. S Patent Application No: 2006/0163463. These references are incorporated herein by reference in their entirety.

Optical tweezers are capable of exerting and measuring forces typically in the range of ˜0.1-100 pN (Moffitt et al. 2008, Ann. Rev. Biochem., 77: 205-228). The use of high refractive index particles can further increase the maximum force (van der Horst et al. 2008, Applied Optics, 47:3196-3202). These forces are typically those encountered inside living cells. As such, the technique is used extensively to study biological processes such as protein folding and force generation by molecular motors and biopolymers (Visscher et al. 1999, IEEE Journal of Selected Topics in Quantum Electronics 2(4):1066-1076).

The application of optical tweezer typically comprises optical trapping a micron-sized, spherical, transparent particle (a “bead”) in a highly focused laser beam. Physically, this is possible because light carries momentum p r with magnitude p=E/c (with E the energy of the photons and c the speed of light) and direction k r (wave vector). When photons change direction due to refraction by the particle, this momentum vector is changed, implying that a force has been exerted on the light wave. Since the particle has exerted a force on the light wave, it follows that (from Newton's third law) that the light exerts an equal but opposite force on the particle (Svoboda and Block 1994, Ann. Rev. Biophys. Biomol. Struct. 23:247-85).

In the example, the inventors monitored the forces applied to the A1/GPIbα polypeptide that is required to extend or stretch out the polypeptide from a ligand-bound state to a ligand-unbonud state with an optical tweezer (see FIG. 8 for the ligand bound and non-ligand bound state of the A1/GPIbα polypeptide). When the A1/GPIbα polypeptide is in a bound state and fully extended, i.e., A1 domain is bound to ligand GPIbα, the polypeptide is of a certain length. When the polypeptide is in the unbound state, i.e., A1 domain is not bound to the ligand GPIbα at the ligand receptor interface, the fully extended polypeptide is longer than in the bound state/fully extended. In a pulling experiment using an optical tweezer, the external force that is applied to stretch the A1/GPIbα polypeptide to opposite ends. When external force that is applied to stretching the A1/GPIbα polypeptide which is in the bound state is greater than the non-covalent interactions holding the A1/GPIbα together, the interaction dissociates, the A1/GPIbα polypeptide take on an unbound state and the polypeptide lengthens slightly with a concomitant decrease in force. FIG. 3B shows a representative trace of a force-extension curve in a pulling experiment. Immediately after dissociation, if no additional force is applied, the A1 domain and GPIbα can re-bind again because they are in close proximity if the force is below that needed for dissociation. The force-extension curve of FIG. 3B shows the point where unbinding and re-binding Occurs.

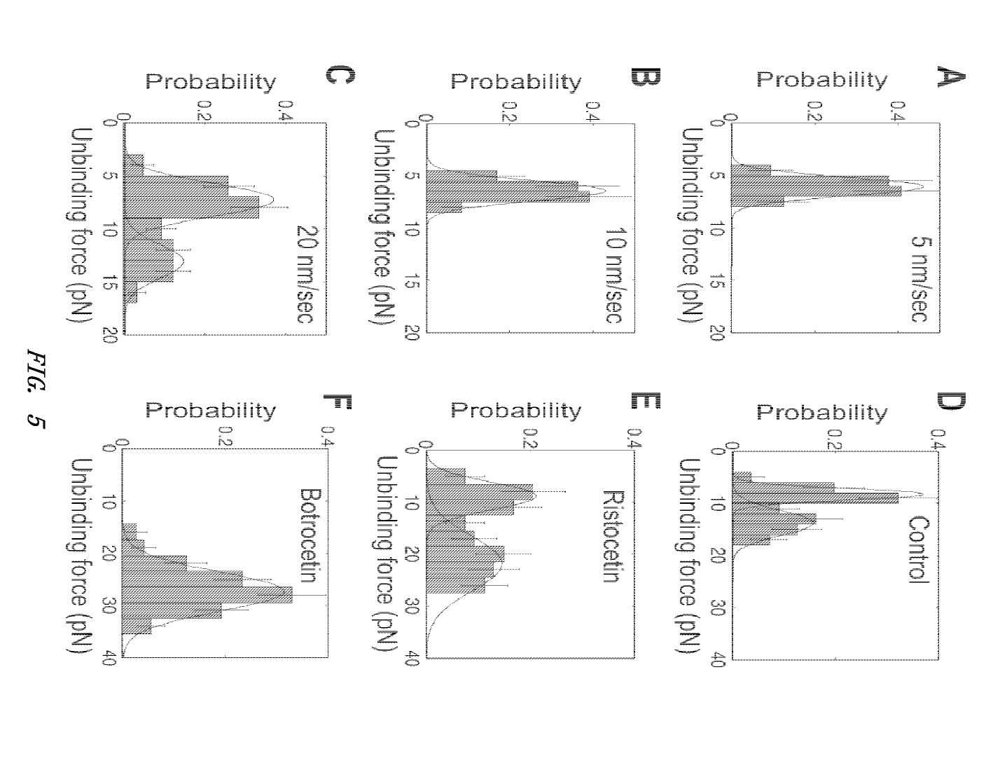

The force at which a receptor-ligand interaction will dissociate is unique to the interaction. It is a function of the non-covalent interactions holding the interaction (typically non-covalent interactions between specific amino acids of the receptor and ligand) and is also a function of the surrounding environment. Therefore, changes in the dissociative force obtained in the presence of an agent can be used as an indicator of whether the agent is a modulator of a receptor-ligand interaction and for identifying such modulators. Modulator(s) of a receptor-ligand interaction can either increase/strengthen/activate or decrease/weaken/inhibit the interaction. In the presence of a strengthener/activator, the dissociative force is greater in the presence of such a modulator compared to in the absence of the modulator. In the presence of a weakener/inhibitor, the dissociative force is lower in the presence of such a modulator compared to in the absence of the modulator. In FIG. 5D-5F, the inventors investigated the dissociative/unbinding force in the absence and presence of two activators of the A1/GPIbα interaction, ristocetin (FIG. 5E) and botrocetin (FIG. 5F). Both strengthener/activators increased the average dissociative/unbinding force for the A1/GPIbα interaction.

In the example, when the A1/GPIbα polypeptide is in a ligand-bound state, is fully extended and stretched by a constant external force, the A1/GPIbα polypeptide oscillates rapidly between the bound and unbound state. By monitoring the extension of the polypeptide, the inventors can determine which state the polypeptide is in; ˜5 nm extension represents the polypeptide in a bound state and ˜15 nm represents the polypeptide in an unbound state (see FIG. 7A-D).When the constant external force applied is below the average dissociative/unbinding force for the A1/GPIbα interaction, the polypeptide spends a greater portion of the time in the ligand-bound state (FIG. 7A). As the force increases towards the average dissociative/unbinding force for the A1/GPIbα interaction, the polypeptide then spends an increasing amount of time in the unbound state. When the force becomes above the average dissociative/unbinding force for the A1/GPIbα interaction, the polypeptide is mostly in the unbound state during the recording time (FIG. 7D). In a series of force clamp experiments using optical tweezer to maintain various constant forces, the inventors were able to demonstrate this oscillation between the bound and unbound state of their A1/GPIbα polypeptide. From multiples of such extension time traces at various constant forces, the inventors can determined the rate of unbinding versus time, the probability of staying bound as a function of the dwell time, and the dissociative/unbinding rates at various constant forces for their A1/GPIbα polypeptide. The dissociative/unbinding rates at various constant forces obtained in the absence of any added molecule, agent or modulator are used as references or controls. The dissociative/unbinding rates at various constant forces obtained in the presence of an added molecule or agent can be used, when compared with a reference or control, as an indicator for identifying and/or determining whether the molecule or agent is a modulator of the receptor-ligand interaction.

Modulator(s) of a receptor-ligand interaction or a bound pair interaction can either increase or decrease the strength of the interaction between a receptor and ligand or between the bound entities. In the former situation, the dissociative/unbinding rate is lower in the presence of such a modulator compared to in the absence of the modulator. Such is a positive modulator. In the latter situation, the dissociative/unbinding rate is greater in the presence of such a modulator compared to in the absence of the modulator. Such is a regative modulator.

In one embodiment, the extending of the ligand-bound-receptor protein occurs with an atomic force microscope (AFM).

In one embodiment, the extending of the ligand-bound-receptor protein occurs with a mobile focused laser light, a cantilever, or a positioner in the AFM.

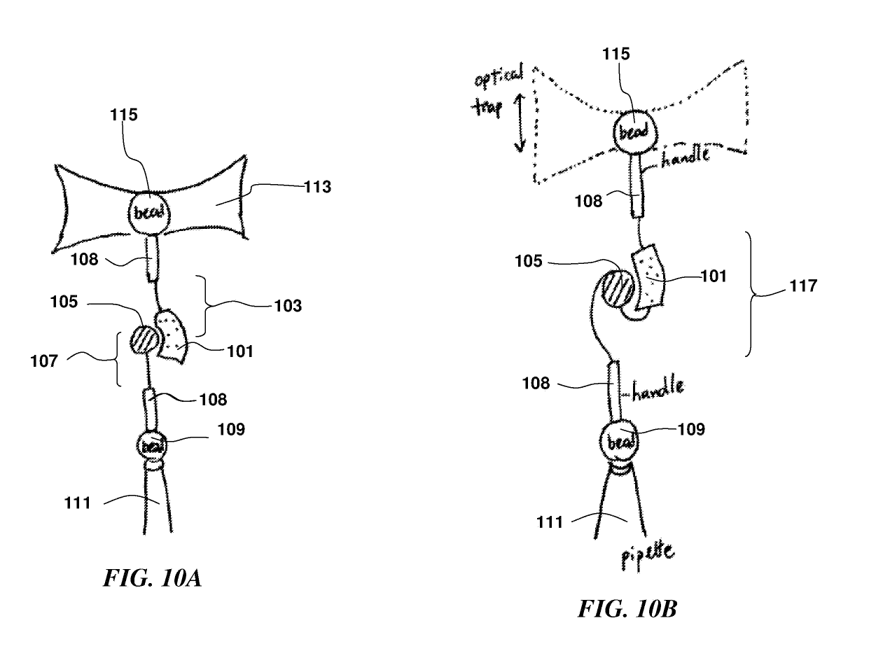

In one embodiment, the extending of the ligand-bound-receptor protein occurs with a mobile focused laser light of an optical tweezer or optical trap microscope. FIG. 10 show exemplary set up for use with an optical tweezer. In FIG. 10A, the ligand-bound-receptor protein is a complex of two separate polypeptides 103 and 107. A receptor polypeptide 103 is coupled to a handle 108 which is then tethered to the surface of a microsphere bead 115. In a similar design, a ligand polypeptide 107 is coupled to a handle 108 which is then tethered to the surface of a microsphere bead 109. The receptor-binding fragment/site 105 of a ligand polypeptide 107 interacts with the ligand-binding fragment/site 101 of a receptor polypeptide 103. In FIG. 10B, the ligand-bound-receptor protein is a single chimeric fusion polypeptide 117 comprising the receptor and ligand binding sites/fragments 105 and 101 respectively. In both FIG. 10A and 10B, the microsphere bead 109 is held static in space by a micropipette 111 while the microsphere bead 115 is manipulated by a mobile focused laser beam 113. By moving the field of the beam 113, the microsphere bead 115 is consequently moved as the bead 115 tries to maintain its desired position within the center of the beam 113. As a result, the ligand-bound-receptor protein can be extended. In other words, the ligand-bound-receptor protein is stretched out away from bead 109 that is held constant in space by the micropipette 111.

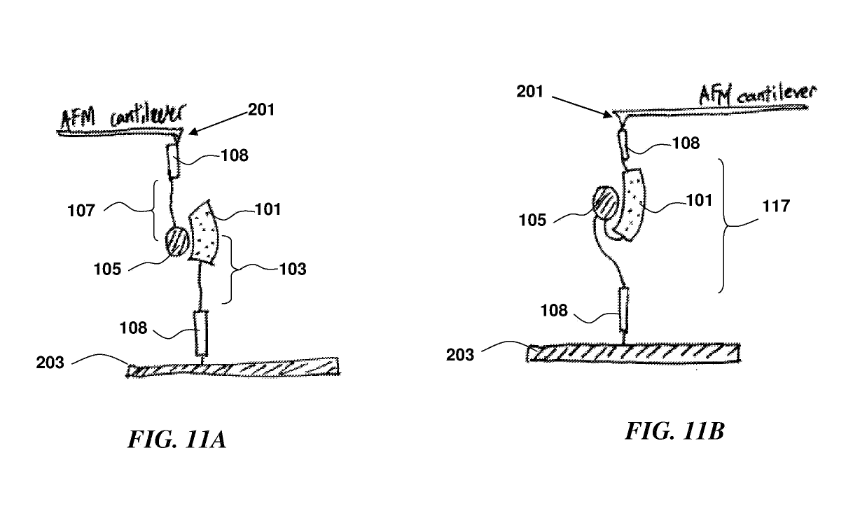

In one embodiment, the extending of the ligand-bound-receptor protein occurs with a cantilever of an AFM. In another embodiment, the extending of the ligand-bound-receptor protein occurs with a movable positioner in an AFM setup. FIG. 11 show exemplary set up for use with an AFM. In FIG. 11A, the ligand-bound-receptor protein is a complex of two separate polypeptides 103 and 107. A receptor polypeptide 103 is coupled to a handle 108 which is then tethered to the surface of a glass slide 203 which is placed on a movable positioner of an AFM set up. In a similar design, a ligand polypeptide 107 is coupled to a handle 108 which is then tethered to the surface of a cantilever tip 201. The receptor-binding fragment/site 105 of a ligand polypeptide 107 interacts with the ligand-binding fragment/site 101 of a receptor polypeptide 103. In FIG. 11B, the ligand-bound-receptor protein is a single chimeric fusion polypeptide 117 comprising the receptor and ligand binding sites/fragments 105 and 101 respectively. The glass slide 203 is held static in space while the cantilever tip 201 is moved. As a result of the cantilever tip 201 movement, the ligand-bound-receptor protein can be extended. In another embodiment, the cantilever is held static and the positioner is moved, e.g., during the constant tension/force experiments.

The atomic force microscope (AFM) or scanning force microscope (SFM) is a very high-resolution scanning probe microscopy, with demonstrated resolution of fractions of a nanometer, more than 1000 times better than the optical diffraction limit. The AFM is one of the foremost tools for imaging, measuring, and manipulating matter at the nanoscale. AFM gathers information by “feeling” the surface with a mechanical probe, e.g., the cantilever. Piezoelectric elements that facilitate tiny but accurate and precise movements on command electronically enable the very precise scanning

The AFM consists of a cantilever with a sharp tip (probe) at its end that is used to scan the specimen surface. The cantilever is typically silicon or silicon nitride with a tip radius of curvature on the order of nanometers. When the tip is brought into proximity of a sample surface, forces between the tip and the sample lead to a deflection of the cantilever according to Hooke's law. Depending on the situation, forces that are measured in AFM include mechanical contact force, van der Waals forces, capillary forces, chemical bonding, electrostatic forces, magnetic forces (see magnetic force microscope, MFM), Casimir forces, solvation forces, etc.

If the tip was scanned at a constant height, a risk would exist that the tip collides with the surface, causing damage. Hence, in most cases a feedback mechanism is employed to adjust the tip-to-sample distance to maintain a constant force between the tip and the sample. Traditionally, the sample is mounted on a piezoelectric tube or a platform that can move the sample in the z direction for maintaining a constant force, and the x and y directions for scanning the sample. Alternatively a ‘tripod’ configuration of three piezo crystals may be employed, with each responsible for scanning in the x, y and z directions. This eliminates some of the distortion effects seen with a tube scanner. In newer designs, the tip is mounted on a vertical piezo scanner while the sample is being scanned in X and Y using another piezo block. The resulting map of the area s=f(x,y) represents the topography of the sample.

The AFM can be operated in a number of modes, depending on the application. In general, possible imaging modes are divided into static (also called contact) modes and a variety of dynamic (or non-contact) modes where the cantilever is vibrated.

In some embodiments of the methods described herein, the A1/GPIbα polypeptide is attached to the tip and the sample surface such that one end of the polypeptide is attached to the tip and the other end of the polypeptide is attached to the sample surface.

For some embodiments of the methods described herein, the AFM is used in force spectroscopy, wherein there are direct measurements of tip-sample interaction forces as a function of the gap between the tip and sample. The result of this measurement is often presented in a form of a force-distance curve.

In one embodiment of the methods described herein, the AFM tip is extended towards and retracted from the surface as the deflection of the cantilever is monitored as a function of piezoelectric displacement. These measurements have been used to measure nanoscale contacts, atomic bonding, Van der Waals forces, and Casimir forces, dissolution forces in liquids and single molecule stretching and rupture forces (Hinterdorfer and Dufrêne, 2006, Nature methods 3(5): 347-55). Forces of the order of a few pico-Newton can now be routinely measured with a vertical distance resolution of better than 0.1 nanometer. Force spectroscopy can be performed with either static or dynamic modes. In dynamic modes, information about the cantilever vibration is monitored in addition to the static deflection (see in “Force measurements with the atomic force microscope: Technique, interpretation and applications”. Surface Science Reports 59: 1-152. 2005).

Methods of using AFM for single molecule measurement are known in the art, e.g., see U.S. Pat. No. 7,441,444; 7,559,261, U.S. Patent Application No. 2008/0289404; T. Hugel and M. Seitz, Macromol. Rapid Commun. 22, 1 (2001); G. Binning, C. F. et al., 1986, Phys. Rev. Lett. 56(9):930; and PCT/EP2008/050075. These references are incorporated herein by reference in their entirety.

In one embodiment, the signal that represents the ligand-bound-receptor protein existing in either a ligand-bound state, in a ligand-unbound state or in a transition between the two states is a force required to dissociate the ligand-receptor interaction and/or produce an increase in extension of the ligand-bound-receptor protein. Examples of such signals are illustrated is FIGS. 3B and 3C, and 5.

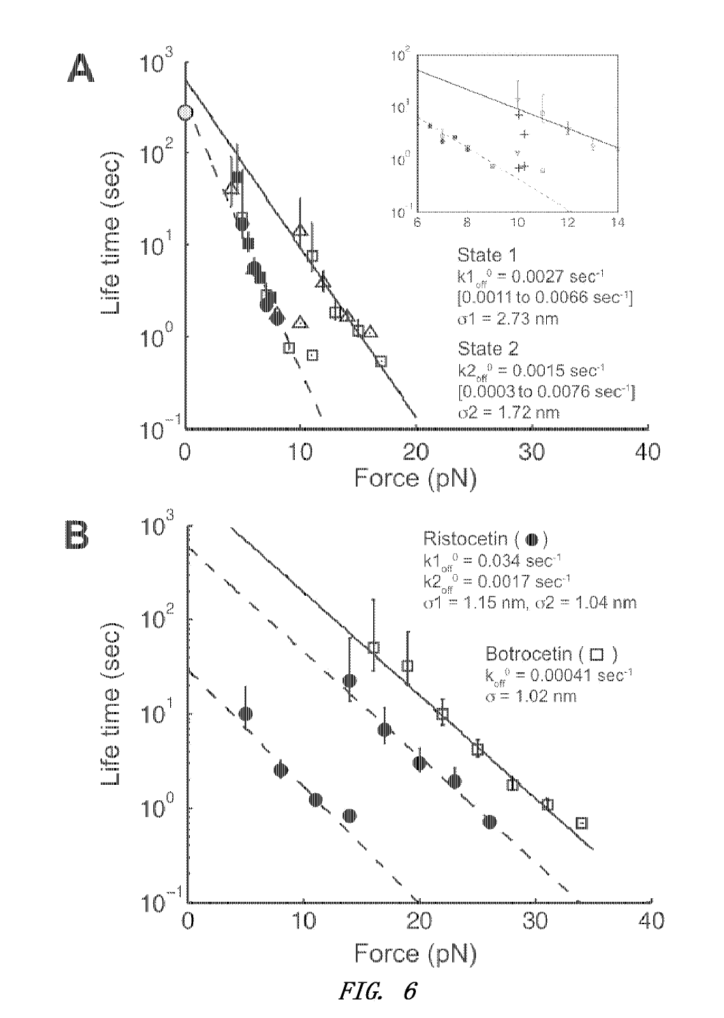

In one embodiment, the signal that represents the ligand-bound-receptor protein existing in either a ligand-bound state, in a ligand-unbound state or in a transition between the two states is a rate of dissociation of the ligand-receptor interaction and/or a dissociation constant of the rate of the ligand-bound-receptor protein held at a constant force. Examples of such signals are illustrated in FIGS. 6 and 7A-D. Methods of computing the rate of dissociation and/or dissociation constant of the rate are well known in the art and are described herein. In FIG. 7, the ligand-receptor interaction is subjected to several different constant forces and the rate of dissociation of the ligand-receptor interaction is computed as the number of dissociation events per unit time for each constant force applied to the A1/GPIbα polypeptide.

In one embodiment, the signal has a positive deviation from a reference signal, which indicates that the agent is positive modulator, i.e., a strengthener, an agonist/activator of the interaction. In another embodiment, the deviation is negative, which indicates that the agent is negative modulator, i.e., a weakener, an antgonist/inhibitor of the interaction.

In one embodiment, the positive deviation of at least 10% from the reference indicates that the modulator is an agonist/strengthener/activator of the receptor-ligand interaction. In other embodiments, the positive deviation is at least 20%, at least 30%, at least 40%, at least 50%, at least 60%, at least 70%, at least 80%, at least 90%, or at least 100% from the reference, including all the whole integers between 10% and 100%.

In one embodiment, the negative deviation of at least 10% from the reference indicates that the modulator is an antagonist/weakener/inhibitor of the receptor-ligand interaction. In other embodiments, the negative deviation is at least 20%, at least 30%, at least 40%, at least 50%, at least 60%, at least 70%, at least 80%, at least 90%, or at least 100% from the reference, including all the whole integers between 10% and 100%.

In one embodiment, the ligand-bound-receptor protein is a fusion chimeric protein comprising (1) a receptor or ligand-binding/functional fragments thereof and (2) a ligand or receptor-binding/functional fragment thereof, wherein the receptor or ligand-binding fragments thereof and the ligand or receptor-binding fragment thereof are fused together in a single polypeptide. In a preferred embodiment, the receptor and ligand or functional fragments thereof are fused in tandem and in close proximity to allow natural receptor-ligand interaction to occur within the fusion polypeptide. As used herein, the term “natural” in reference to the receptor-ligand interaction refers to the interaction between a receptor molecule and its ligand that occurs in nature wherein the receptor and its ligand are in their natural environments and are not fused to each other in a single polypeptide.

In one embodiment, in the single fusion polypeptide comprising a receptor or ligand-binding fragments thereof and a ligand or receptor-binding fragment thereof, the receptor and ligands or functional fragments are separated by at least one linker moiety. The linker moiety functions as a spacer between the receptor portion and the ligand portion of the fusion polypeptide. In preferred embodiments, the linker moiety does not interfere with the natural receptor and ligand interaction.

In one embodiment, the linker moiety is a spacer linker peptide. In one embodiment, the spacer peptide linker has the sequence of

TGGPTIKPPKPPKPAPNLLGGPDKTHTKPPKPAPELLGGPGTG (SEQ. ID. NO: 1). This peptide is modified from the hinge regions of murine IgG2a and human IgG1, with cysteine residues either removed or substituted with proline.

In one embodiment, the spacer linker peptide has at least one amino acid residue and up to about 200 amino acid residues. In other embodiments, the spacer linker peptide has 2, 3, 4, 5, 6, 7, 8, 9, 10, . . . 15, . . . 20, . . . 30, . . . 40, . . . 50, . . . 60, . . . 70, . . . 80, . . . 100, . . . 110, . . . 125, . . . 150, . . . 175, . . . 200 amino acid residues, including all the whole integers between the number 10 and 200.

In one embodiment, the fusion chimeric protein is made by recombinant methods known in the art, e.g., the fusion chimeric protein in encoded by and expressed from a fusion chimeric nucleic acid comprising nucleic acid of (1) a receptor or ligand-binding fragments thereof and (2) a ligand or receptor-binding fragment thereof, wherein the nucleic acid sequences of (1) and (2) are in tandem and in frame with respect to each other, so that a fusion chimeric polypeptide is transcribed and translated from the fusion chimeric nucleic acid. In one embodiment, the fusion chimeric nucleic acid further comprises a nucleic acid sequence encoding a spacer linker peptide described herein, wherein the spacer linker peptide encoding nucleic acid sequence is placed between the nucleic acid sequence encoding the receptor and the nucleic acid sequence encoding the ligand. In preferred embodiments, the linker moiety does not interfere with the natural receptor and ligand interaction.

In another embodiment, the fusion chimeric protein is made by linking two separate and independent polypeptides, the first polypeptide being a receptor or a ligand-binding fragment thereof and the second polypeptide being a ligand or a receptor-binding fragment thereof. The first and the second polypeptide can be recombinant proteins that are separately expressed and purified. Then, they are physically linked together by any methods knows in the art, e.g., using chemical crosslinkers such as xylyl dithiol (XYL). XYL=HS—CH₂—C6H4-CH₂—SH and PEG linkers. In some embodiments, the two polypeptides can also be linked by non-covalent bonds located at non-ligand binding/non receptor-binding regions of the polypeptides. In some embodiments, the two polypeptides are also linked by covalent bonds located at non-ligand binding/non receptor-binding regions of the polypeptides. In preferred embodiments, the linker moiety does not interfere with the natural receptor and ligand interaction. In a preferred embodiment, the first and second polypeptides are linked in tandem and in close proximity to allow natural receptor-ligand interaction to occur within the fusion chimeric protein.

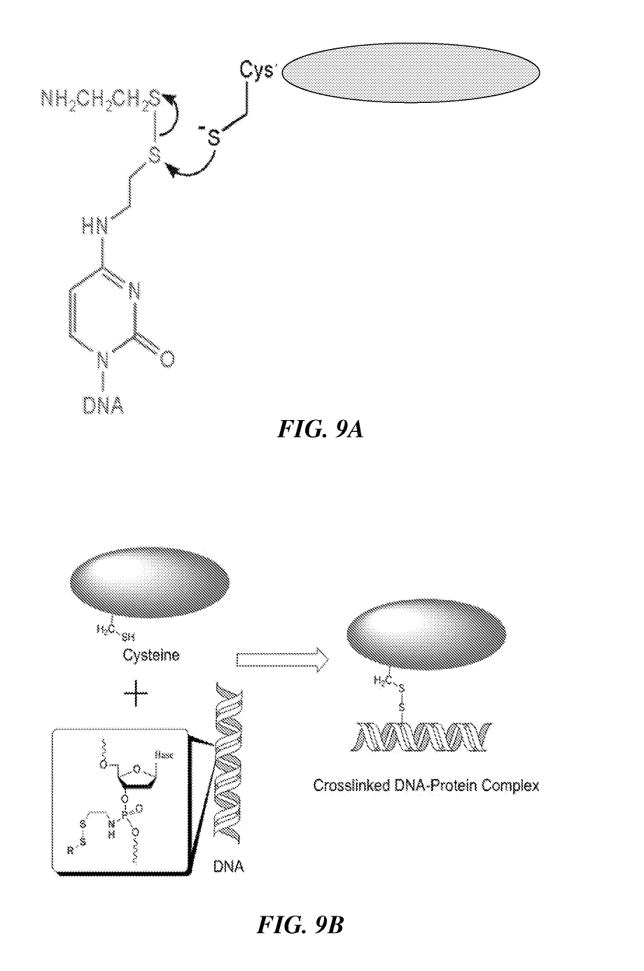

In one embodiment, the fusion chimeric protein has extraneous amino acid residues at one or both termini for the purposes of coupling with a handle, e.g., a dsDNA. The handle allows tethering of the fusion chimeric protein to a solid surface such as a microsphere or a glass silde (see FIGS. 10A and 11A). In a preferred embodiment, the extraneous amino acid residues comprise at least one cysteine residue for the purpose of forming disulfide bridges with a handle, e.g., a thiol tethered dsDNA as shown it FIG. 9.

In one embodiment, the ligand-bound-receptor protein is a complex of two independent polypeptides wherein a first polypeptide comprises a receptor or ligand-binding fragments thereof and a second polypeptide comprises a ligand or receptor-binding fragment thereof; and wherein the two polypeptides complex is by way of the ligand-receptor interaction.

In one embodiment, the ligand is a natural ligand of the receptor. For example, GP1bα is the natural ligand of the VWF A1 domain.

In one embodiment, the ligand is an artificial ligand of the receptor, for example a synthetic drug or viral/pathogen component.

In one embodiment, the fusion chimeric protein is tethered to at least one solid surface. In one embodiment, the ligand-bound receptor is tethered to at least a solid surface. In one embodiment, the tethering occurs at the terminus of the molecule. Preferably, the tethered end of the molecule, i.e., the fusion chimeric protein or the ligand-bound receptor, is distal and far away from the interaction/binding portion, so that the tethering does not interfere with the interaction of interest. In one embodiment, the solid surface is a microsphere or a glass slide. For example, the surface of a glass slide that is used on the mobile positioner of an AFM (see FIG. 11) or a microsphere for manipulation with an optical tweezer as shown in FIG. 10.

In one embodiment, both amino and carboxyl ends of the fusion chimera protein are tethered to separate solid surfaces using handles for use with the optical tweezer or an AFM. In one embodiment, both amino and carboxyl ends of the ligand-bound receptor are tethered to separate solid surfaces by handles for use with an optical tweezer or an AFM.

In one embodiment, only one end of the protein is coupled to a handle for tethering to a surface for use with an optical tweezer or atomic force microscope. For example, wherein the ligand-bound-receptor protein is a complex of two independent polypeptides, only one end of each of the two polypeptides is coupled to a handle for use with an optical tweezer or an AFM. Preferably, the end used for coupling to a handle is not the end required for complexing with the other polypeptide and/or does not interfere with complexing with the other polypeptide. In one embodiment, the end that is coupled to the handle is furthest from the interaction.

In one embodiment, the handle is a double-stranded DNA (dsDNA). In some embodiments, the dsDNA comprises thiol-tethered bases. An example of a thiol-tethered base is a modified cytosine base with a thiol tether introduced at the N4 position (see FIG. 9A). A modified cytosine can interact with the N- and C-terminus cysteine residues in the fusion protein and form a disulfide crosslink between the protein and the modified DNA. For example, replacement of O6-alkylguanaine with a modified cytosine (C*) bearing an O4-thiol tether in a duplex DNA provides a reactive disulfide group that can be attacked by cysteine residues (see FIG. 9A). Methods of introducing thiol-tethered bases into DNA and methods of use with disulfide linking to proteins are known in the art, for example, as described in Mishina Y. 2004, Nucleic Acids Research, 2004, 32: 4 1548-1554; Erica M. Duguid et al., 2003, Chemistry & Biology, 10:827-835; Y. Z. Xu, et al., J. Org. Chem., 1992, 57:3839-3845; A. M. MacMillan and G. L. Verdine, J. Org. Chem. 1990, 55;5931-5933; and A. M. MacMillan and G. L. Verdine, Tetrahedron 1991, 14:2603-2616; Banerjee, A., Santos, W. L., and Verdine, G. L. Science 2006, 311:1153-1157 and U.S. Pat. No. 5,578,718. These references are incorporated herein by reference in their entirety.

As an exemplary, the thiol-tethered oligonucleotide having the sequence of 5′-TACCGCAGCCATCAGAGT-3′ (SEQ. ID. NO: 2) can be synthesized using any methods known in the art and described herein. The thiol tether can be attached to the backbone phosphate between bases 11 and 12. Double-stranded DNA can be formed by mixing the two complementary oligonucleotides 1:1 in a buffer containing 25 mm NaCl and 15 mm Tris-HCl (pH 7.5). The mixture was heated to 85° C. and then cooled slowly to room temperature. Cross-linking reactions can be performed by mixing proteins described herein (1 μm) with thiol-tethered double-stranded DNA (2 μm) in 15 μl of reaction buffer (30 mm Tris-HCl (pH 7.5), 30 mm NaCl, and 10 μm dithiothreitol (DTT)) for 1 h at room temperature in the presence or absence of 2 mm DTT. The reaction is stopped by capping the free thiol groups with S-methyl methane thiosulfate (40 mm). The quenched reaction mixtures can be analyzed on a 10% SDS-polyacrylamide gel under nonreducing conditions. The gel can stained using SIMPLYBLUE™ Safe Stain (INVITROGEN™) overnight and destained in water.

In one embodiment, the ligand-bound-receptor protein, the receptor-ligand pair, a bound pair or interacting proteins described herein is not tethered to the at least one solid surface by way of a dsDNA.

In one embodiment, the receptor-ligand pair is VWF A1 domain and GP1bα subunit.

In one embodiment, the receptor-ligand pair is α4β7-MAdCAM-1.

The adhesive interaction between leukocyte integrin α4β7 and its endothelial ligand, mucosal addresin cell adhesion molecule-1 (MAdCAM-1) mediates the rolling and firm adhesion of leukocytes to the high endothelial venules of mucosal tissues. Over-activation of this adhesive mechanism leads to chronic inflammation. A key property of integrin α4β7 is that on resting leukocytes, the integrin α4β7 mediates rolling. Upon leukocyte activation by chemokines, integrin α4β7 is induced into a high affinity conformation and thus mediate firm adhesion.

MAdCAM-1 belong to a family of mucosal cellular adhesion proteins and members adopt an immunoglobulin-like beta-sandwich structure, with seven strands arranged in two beta-sheets in a Greek-key topology. They are essential for recruitment of lymphocytes to specific tissues. Mucosal cellular adhesion proteins are cell adhesion molecules expressed on the endothelium in mucosa that guide the specific homing of lymphocytes into mucosal tissues. MAdCAM-1 belongs to a subclass of the immunoglobulin superfamily (IgSF), the members of which are ligands for integrins (PUBMED:9655832; PUBMED:11807247; PUBMED:9655832) (Dando J, et al., Acta Crystallogr D Biol Crystallogr. 2002;58:233-241; Tan K. et al. Structure. 1998, 15;6(6):793-801).

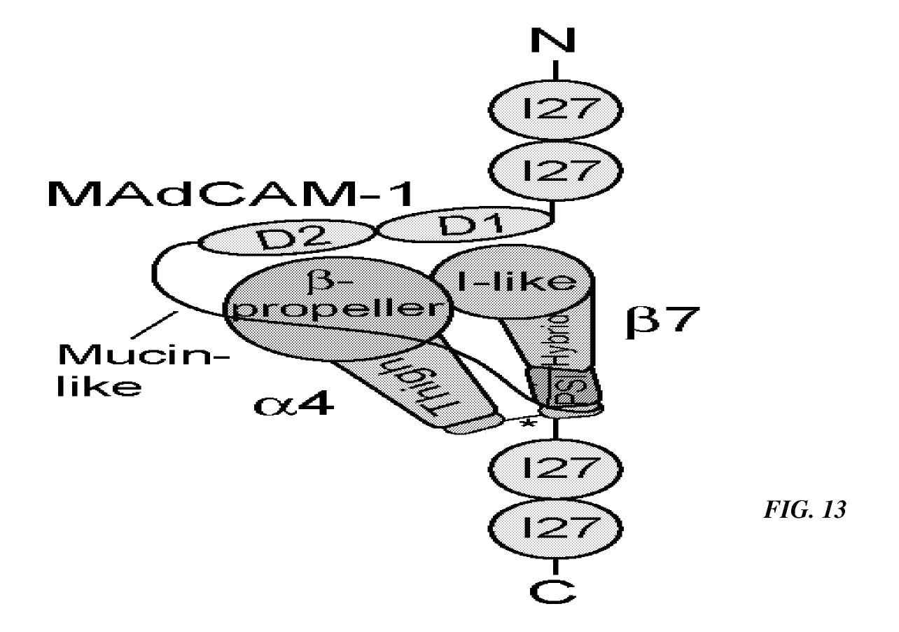

The α4β7-MAdCAM-1 pair construct contains the following modules: two titin I27; a short linker; MAdCAM-1 including its mucin-like domain; αβ7 head (residue 1-465); and two titin I27 from the N- to the C-terminus. The α4 head (propeller+thigh) is expressed as a separate chain, and is associated during the biosynthesis of the other headpiece constructs. The C-terminal I27 module contains two cysteines that allow a covalent attachment of the protein to a gold-coated substrate (see FIG. 13). This construct system is ideal for an AFM study. The mucin-like domain of MAdCAM-1 is long (125 residues) and flexible, which allows intramolecular binding between D1-D2 of MAdCAM-1 and α4β7 headpiece.

In one embodiment, the receptor-ligand pair is αL integrin I domain—ICAM-1(D1+D2). In another embodiment, the receptor-ligand pair is αL integrin I domain—ICAM-3 (D1).

Integrin alpha L (also known as antigen CD11A (p180); lymphocyte function-associated antigen 1; alpha polypeptide; LFA-1; LFA1A; ITGAL) is an alpha L chain protein which functions in the immune system. It is involved in cellular adhesion. It is the target of the drug efalizumab.

Integrins are heterodimeric integral membrane proteins composed of an alpha chain and a beta chain. The I-domain containing alpha integrin combines with the beta 2 chain (ITGB2) to form the integrin lymphocyte function-associated antigen-1 (LFA-1), which is expressed on all leukocytes (Larson RS et al., 1989, J. Cell Biol. 108: 703-12; Shimaoka, Motomu, et al., 2003, Cell 112: 99-111; Curr. Opin. Cell Biol. 2006 18:579-86). The I domain encompasses amino acid residues 145-324 of the 1145 amino acid long mature αL integrin subunit protein (amino acid residues 26-1170 of GENBANK™ Accession No. NP_{—}002200). The ligand binding site of the I domain, known as a metal ion-dependent adhesion site (MIDAS), exists as two distinct conformations allosterically regulated by the C-terminal α7-helix. LFA-1 plays a central role in leukocyte intercellular adhesion through interactions with its ligands, ICAMs 1-3 (intercellular adhesion molecules 1 through 3), and also functions in lymphocyte co-stimulatory signaling. Two transcript variants encoding different isoforms have been found for this gene.

Intercellular adhesion molecules (ICAMs) are part of the immunoglobulin superfamily. They are important in inflammation, immune responses and in intracellular signalling events. The ICAM family consists of five members, designated ICAM-1 to ICAM-5. They are known to bind to leucocyte integrins CD11/CD18 during inflammation and in immune responses. In addition, ICAMs can exist in soluble forms in human plasma, due to activation and proteolysis mechanisms at cell surfaces.

All ICAM proteins are type I transmembrane glycoproteins, contain 2-9 immunoglobulin-like C2-type domains, designated as D domains, and bind to the leukocyte adhesion LFA-1 protein. This protein is constitutively and abundantly expressed by all leucocytes and is an important ligand for LFA-1 in the initiation of the immune response. It functions not only as an adhesion molecule, but also as a potent signaling molecule.

Intercellular adhesion molecule-1″ or “ICAM-1”, i.e. GENBANK™ Accession Nos. NM_{—}000201, NP_{—}000192, is the ligand for αLβ2 integrin, and its N-terminal domain (D1) binds to the αL I domain through the coordination of ICAM-1 residue Glu-34 to the MIDAS metal. It is also a ligand for fibrinogen, human rhinovirus and Plasmodium falciparum-infected erythrocytes.

The intercellular adhesion molecule 1 (also known as ICAM-1, BB2; CD54; P3.58; ICAM1) is a cell surface glycoprotein which is typically expressed on endothelial cells and cells of the immune system. It binds to integrins of type CD11A/CD18, or CD11B/CD18.

The ICAM-1 molecule consists of five Ig-like domains (D1-D5), a short transmembrane region, and a small carboxyl-terminal cytoplasmic domain. The second, third, and fourth Ig domains are heavily N-glycosylated with four potential sites in D2, two in D3, and two in D4. The normal adhesive ligands are two integrins, leukocyte function-associated antigen (LFA-1, CD11a/CD18) (7-9), and macrophage-1 antigen (Mac-1, CD11b/CD18). Adhesion between ICAM-1 and LFA-1 is primarily between the D1 domain and the insertion (I)-domain of the respective molecules, whereas adhesion between ICAM-1 and Mac-1 is between the D3 domain and the I-domain. The 3-dimensional atomic structure of the tandem N-terminal Ig-like domains (D1 and D2) of ICAM-1 has been determined to 2.2A resolution and fitted into a cryoelectron microscopy reconstruction of a rhinovirus-ICAM-1 complex (B. J. Kolatkar, et al., 1998, Proc. Natl. Acad. Sci. U S A. 95:4140-5). Two other molecules, ICAM-2 and ICAM-3, have at least 30% sequence identity with ICAM-1 and have similar adhesive properties. ICAM-1 and ICAM-2 normally have low expression levels, whereas ICAM-3 is more abundant in resting monocytes and lymphocytes. Fibrinogen can also bind to domain D1 of ICAM-1, mediating leukocyte adhesion to vascular endothelium Unlike many other integrin receptors, ICAM-1 does not possess an Arg-Gly-Asp (RGD) motif, but has a larger, more extended binding surface. The D1-D2 encompasses amino acid residues 1-185 of GENBANK™ Accession Nos. NM_{—}000201, NP_{—}000192.

ICAM-3 (CD50) contains five Ig-like domains and binds to leukocyte integrins CD11A-D/CD18. The protein plays an important role in the immune response and perhaps in signal transduction (Neelamegham S, et al., 2000, J Immunol., 164:3798-805).

In one embodiment, the receptor-ligand pair is fimH pilin+lectin domain−N-linked carbohydrate.

FimH is a bacterial adhesion protein found on the tip of several microganisms fimbriae or pili., e. g. uropathogenic Escherichia coli type 1 (Jones, C. H., 1995, Proc. Natl. Acad. Sci. USA 92: 2081-2085; S. G. Stahlhut, et al., 2009, Journal of Bacteriology, 6191:592-6601). The fimH protein is critical for mediating bacterial colonization and invasion of bladder and urinary tract epithelium.

Fim H proteins adopt a secondary structure consisting of a beta sandwich, with nine strands arranged in two sheets in a Greek key topology. They are predominantly found in bacterial mannose-specific adhesins, since they are capable of binding to D-mannose (Hung C S, et al., 2002, Mol. Microbiol. 44:903-915; PDB entry: 2vco).

FimH contains two Ig-like domains—a pilin domain that is incorporated into the pilus, and a mannose-binding lectin domain that interacts with mannosylatedglycans on epithelial surfaces; the mannose-binding lectin domain (Ld) (1-156 amino acid) and the fimbria-incorporating pilin domain (Pd) (160-273 amino acid), which are connected via a 3-amino acid interdomain linker peptide chain. The FimH-mannose interaction has been shown to increase in strength under shear force. This adaptation helps aid colonization in the bladder and urinary tract in spite of this high flow environment.

In one embodiment, the receptor-ligand pair is fimH-fimG (Jones, C. H., 1995, Proc. Natl. Acad. Sci. USA 92: 2081-2085).

In one embodiment, the ligand is not a protein or peptide. In some embodiment, the ligand is a carbohydrate, e.g., a mannose containing carbohydrate.

In one embodiment, the receptor-ligand pair is glycosylated, e.g., N-glycosylated or O-glycosylated. In other embodiments, the receptor-ligand pair or bound pair of the methods described herein is modified. For example, polypeptides can be post-translationally modified, e.g., phosphorylated, glycosylated, deamidation, carbamylation, sulfation, selenoylation, phosphopantetheinylation, isoprenylation or prenylation, alkylation, acylation, and amidation to name a few.

It is contemplated that the methods described herein be used for the protein interactions that are associated with disease such as cancer and autoimmune diseases. For example, the screening method can be used for finding negative modulator of B-lymphocyte antigen CD20 or CD20 with its receptor in vivo. CD20 is found on B-cell lymphomas, hairy cell leukemia, and B-cell chronic lymphocytic leukemia. It is also found on skin/melanoma cancer stem cells. For example, the screening method can be used for finding positive modulator of the interaction of tositumomab with CD20. Tositumomab is an anti-CD20 antibody used in the treatment of non-Hodgkins lymphoma. A positive modulator of such interaction can improve the efficacy of tositumomab treatment and may also permit less tositumomab be used in the treatment. The option to reduce the amount of a treatment drug is useful especially when there the drug has undesirable side effects. Other monoclonal antibodies that are used for autoimmune diseases include infliximab and adalimumab, which are effective in rheumatoid arthritis, Crohn's disease and ulcerative Colitis by their ability to bind to and inhibit TNF-α. Basiliximab and daclizumab inhibit IL-2 on activated T cells and thereby help preventing acute rejection of kidney transplants. Omalizumab inhibits human immunoglobulin E (IgE) and is useful in moderate-to-severe allergic asthma. The screening method described herein can be used for finding negative modulator of the interactions between TNF-α with its receptor, IL-2 with its receptor; and IgE with its receptor. In addition, the screening method can be used for finding positive modulator of the interactions of infliximab and adalimumab with TNF-α, basiliximab and daclizumab with IL-2, and omalizumab with IgE. Other relevant interactions for which the screening method is applicable are shown in Table 1.

### Definitions of Terms

By the term “screening” in relation to a modulator of the interaction between two binding partners described herein the term means the act of evaluating or testing a number of molecules to identify those with a particular set of attributes or characteristics, specifically, capability of changing the strength of the interaction between two binding partners, for example, between a receptor and ligand pair. The change in the strength of the interaction can corresponds to a decrease or an increase in the strength of interaction. Changes in the strength of the interaction lead to an increase or a decrease in the dissociation forces/rates at as determined by the method described herein.

As used herein, the term “modulator” refers to any entity that changes the interaction between two binding partners, for example, between a receptor and ligand pair. The change is either a decrease or an increase in the strength of interaction such that more force or less force is required to disrupt or break the interaction in the presence of the modulator; or the dissociation rate constant of the interaction between the two molecule is changed in the presence of the modulator, i.e., greater or less than in the absence of the modulator.

As used herein, the term “ interaction” when used in the context of a receptor and its ligand refers to the binding between the receptor and its ligand as a result of the non-covalent bonds between the ligand-binding site (or fragment) of the receptor and the receptor-binding site (or fragment) of the ligand. In the context of two entities, e.g., molecules or proteins, having some binding affinity for each other, the term “interaction” refers to the binding of the two entities as a result of the non-covalent bonds between the two entities. A term “interaction”, “complexing” and “bonding” are used interchangeably when used in the context of a receptor and its ligand and in the context of two binding entities.

As used herein, the term “comprising” means that other elements can also be present in addition to the defined elements presented. The use of “comprising” indicates inclusion rather than limitation.

As used herein, the term “a receptor and ligand pair” and “bound pair” refers to at least two binding partners that, when mixed together or are in very close proximity and under conditions that permit binding, will normally interact and bind to each other in the absence of other molecules. In nature, the two binding partners are two separate and independent molecules, e.g., proteins and polypeptides. The binding of such pair would produce a standard saturation binding curve in binding studies wherein when the concentration of one molecule is held constant and the concentration of the other molecule is increased. Such binding curves are well known to a skilled artisan. In one embodiment of the context of the methods described herein, “a receptor and ligand pair” refers to a chimeric fusion polypeptide wherein the regions responsible for the binding on the two binding partners are found on a single polypeptide, e.g., the VWF A1 domain/GPIbα polypeptide described herein.

As used herein, the term “ligand-bound-receptor protein” refers to a receptor protein or ligand-binding fragment thereof that is currently bound with its ligand or receptor-binding fragment thereof. For example, the receptor is Von Willebrand factor (VWF) with the platelet glycoprotein Ib a (GPIbα) in the ligand. The GPIbα ligand binding fragment of VWF is the A1 domain. In a buffered solution, when the A1 domain is bound to and occupied by GPIbα, the VWF A1 domain/GPIbα polypeptide is a “ligand-bound-receptor protein”. A “ligand-bound-receptor protein” is in a “ligand-bound state” wherein the ligand is bound to or occupying the ligand bind site of the receptor. In some embodiments, the receptor or ligand binding fragments may not be ligand bound or occupied. In such instances, the receptor is in a “ligand-unbound state”. It is contemplated that in a “ligand-bound-receptor protein”, the ligand, the receptor and the relevant respective fragments thereof can be separate and independent protein molecules, and they are not a chimera fusion protein such as the VWF A1 domain/GPIbα polypeptide described in the example.

The term “contacting” or “contact” as used herein in connection with contacting a ligand-bound-receptor protein with an agent as disclosed herein, includes mixing proteins to a solution which comprises an agent.

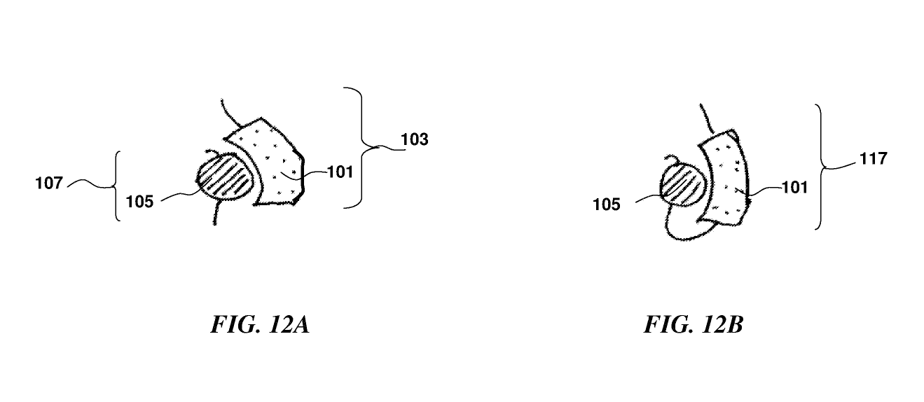

As used herein, the term “extending” when used in the context of ligand-bound-receptor protein refers to “stretching out” the ligand-bound-receptor protein. The goal can be to overcome and break the non-covalent forces at the receptor-ligand interaction that is holding the ligand and the receptor together. Alternatively, the goal is not to break the receptor-ligand interaction but to maintain a specific tension in the ligand-bound-receptor protein when the protein is stretched in opposite directions. In some embodiments, “extending” refers to pulling the ends of the ligand-bound-receptor protein in separate and opposite directions by methods described herein to achieve the goal of overcoming and breaking the non-covalent forces holding the ligand and the receptor together. In other embodiments, “extending” refers to pulling the ends of the ligand-bound-receptor protein in separate and opposite directions by methods described herein such that the protein is “stretched out” but the ligand-receptor interaction remains intact and there is a specific tension within the protein. In the embodiment where a single chimeric fusion polypeptide comprises the ligand-bound-receptor protein, i.e., a single polypeptide has both the receptor-and the ligand-binding sites, “extending” such a polypeptide means moving the amino and the carboxyl terminus of the polypeptide away from each other (see FIGS. 10B, 11B and 12B). In the embodiment where the ligand-bound-receptor protein is a complex two separate protein, that of a receptor polypeptide and a ligand polypeptide held together by the interaction of the receptor with the ligand-binding sites, “extending” such a complex refers to moving the terminus distal to the receptor-binding site of the ligand polypeptide in an opposite direction from the terminus distal to the ligand-binding site of the receptor polypeptide (see FIGS. 10A, 11A and 12A).

As used herein, the term “chimeric fusion protein” or “chimeric fusion polypeptide” refers to a protein created by joining two protein coding genes or two proteins/peptides together. In the laboratory, this is achieved through the creation of a fusion gene which is done through the removal of the stop codon from a DNA sequence encoding the first protein and then attaching the DNA sequence of the second protein in frame after the DNA sequence encoding the first protein. The resulting fusion DNA sequence will then be expressed by a cell as a single protein. In a fusion protein, the two proteins that are joined together with a linker or spacer peptide added between the two proteins. This linker or spacer peptide often contain protease cleavage site to facilitate the separation of the two proteins after expression and purification. The making of fusion protein as a technique known in the art.

“Polypeptide” and “protein” are used interchangeably herein to refer to a polymer of amino acid residues. In one embodiment, the terms apply to amino acid polymers in which one or more amino acid residue is an artificial chemical mimetic of a corresponding naturally occurring amino acid. In another embodiment, the terms apply to amino acid polymers with naturally occurring amino acid polymers. In another embodiment, the terms apply to amino acid polymers with non-naturally occurring amino acid polymer. In another embodiment, the terms apply to amino acid polymers with naturally occurring amino acid polymers and non-naturally occurring amino acid polymer. “Polypeptide” and “protein” further refer to amino acids joined to each other by peptide bonds or modified peptide bonds, i.e., peptide isosteres, and can contain modified amino acids other than the 20 gene-encoded amino acids. The term “polypeptide” also includes polypeptide fragments, motifs and the like. In one embodiment, the terms apply to glycosylated polypeptides.

As used herein, the term “agent” refers to any entity. An agent can be selected from a group comprising: chemicals; small molecules; nucleic acid sequences; nucleic acid analogues; proteins; peptides; aptamers; antibodies; or functional fragments thereof. A nucleic acid sequence can be RNA or DNA, and can be single or double stranded, and can be selected from a group comprising: a nucleic acid encoding a protein of interest; oligonucleotides; and nucleic acid analogues; for example peptide-nucleic acid (PNA), pseudo-complementary PNA (pc-PNA), locked nucleic acid (LNA), etc. Such nucleic acid sequences include, but are not limited to nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. An agent can be applied to a solution, where it contacts the ligand-bound receptor protein. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

As used herein, the term “antagonist” is an entity that will inhibits, impedes and/or weaken the ligand-receptor pair interaction. Such an antagonist brings about reduced binding affinity, as indicated by, for example, K_{d} value, (dissociation constant) for a ligand-receptor pair interaction. For example, an antibody that blocks the interaction of a protease and PAR receptor protein, and there by preventing the cleavage-activation of the PAR signaling pathway. Such an antagonist brings about a decrease in the disruption force needed to dissociate the receptor-ligand interaction. Such an antagonist also brings about an increase in the rate of dissociation the receptor-ligand interaction when the complex is held at a constant force. The terms “antagonist”, “inhibitor” and “weakener” are used interchangeably.

As used herein, “agonist” means an entity that will promotes, stimulates, activates, enhances and/or strengthens the ligand-receptor pair interaction. Such an agonist brings about greater binding affinity, as indicated by, for example, K_{d} value, (dissociation constant) for a ligand-receptor pair interaction. Such an agonist brings about an increase in the disruption force needed to dissociate the receptor-ligand interaction. Such an agonist also brings about a decrease in the rate of dissociation the receptor-ligand interaction when the complex is held at a constant force. For example, a RARγ specific agonists bind to the RARγ receptor at significantly lower concentrations (>10-fold selectivity, such as 50-fold to 100-fold selectivity) than the RARα and RARβ receptors. See, e.g., U.S. Pat. No. 6,300,350; WO 01/080894. For example, CD 1530 (chemical name 4-(6-Hydroxy-7-tricyclo[3.3.1.13,7]dec-1-yl-2-naphthale nyl) benzoic acid) is a potent and selective RARγ receptor agonist (K_{d} values are 150, 1500 and 2750 nM for RARγ, RARβ and RARα receptors respectively). CD 1530 activates transcriptional activity (AC50=1.8 nM). For example, in the A1 domain/GP1bα interaction, ristocetin and botrocetion are agonists of this interaction as the interaction is strengthen as indicated by an increase of the unbinding forces needed (see FIG. 5). The terms “agonist”, “activator” and “strengthener” are used interchangeably.

By the wording “functional fragments” when used in reference to a receptor refers to the portion or part of a full-length receptor polypeptide that is responsible and/or involved in binding its ligand. A “functional fragment” of a receptor is not the full-length receptor polypeptide. The “functional fragments” of receptor is also referred to as the “ligand-binding fragment” of the receptor.

By the wording “functional fragments” when used in reference to a ligand refers to the portion or part of a full-length ligand polypeptide that is responsible and/or involved in binding its receptor. A “functional fragment” of a ligand is not the full-length ligand polypeptide. The “functional fragments” of a ligand is also referred to as the “receptor-binding fragment” of the ligand.

As used herein, the term “fragment” in the context of a polypeptide refers to a polypeptide that is not a full-length polypeptide as it is naturally encoded in the genome, and therefore the length of amino acid sequence of the fragment of the polypeptide is shorter than the length of the full-length polypeptide. Such fragments may be selected from, but not limited by examples of naturally occurring isoforms of the polypeptides, proteolytic fragments of the above polypeptides, truncated proteins resulting from nonsense mutations, corresponding recombinant polypeptides or fusion proteins containing amino acid sequences derived from the polypeptides.

### Production of Polypeptides, Functional Fragments and Peptide Linkers

Receptor and/or ligand polypeptides, functional fragments thereof, and spacer linker peptides described herein can be provided by any suitable conventional method known in the art. The polypeptides, functional fragments thereof and spacer linker peptides can be, for example, chemically synthesized (for example, see Creighton, “Proteins: Structures and Molecular Principles,” W.H. Freeman & Co., NY, 1983), or, perhaps more advantageously, produced by recombinant DNA technology as described herein. For additional guidance, skilled artisans can consult Sambrook and Russel (“Molecular Cloning, A Laboratory Manual,” Cold Spring Harbor Press, Cold Spring Harbor, N.Y., 2001, 3^{rd} edition), and, particularly for examples of chemical synthesis Gait, M. J. Ed. (“Oligonucleotide Synthesis,” IRL Press, Oxford, 1984). These references are incorporated herein by reference in their entirety.

Polypeptides, functional fragments thereof and spacer linker peptides can be chemically synthesized and purified by biochemical methods that are well known in the art such as solid phase peptide synthesis using t-Boc (tert-butyloxycarbonyl) or FMOC (9-flourenylmethloxycarbonyl) protection group as described in “Peptide synthesis and applications” in Methods in molecular biology Vol. 298, Ed. by John Howl; “Chemistry of Peptide Synthesis” by N. Leo Benoiton, 2005, CRC Press, (ISBN-13: 978-1574444544); “Chemical Approaches to the Synthesis of Peptides and Proteins” by P. Lloyd-Williams, et. al., 1997, CRC-Press, (ISBN-13: 978-0849391422); Methods in Enzymology, Volume 289: Solid-Phase Peptide Synthesis, J. N. Abelson, M. I. Simon, G. B. Fields (Editors), Academic Press; 1st edition (1997) (ISBN-13: 978-0121821906); U.S. Pat. Nos. 4,965,343, and 5,849,954. These references are incorporated herein by reference in their entirety.

Commercial peptide synthesizing machines are available for solid phase peptide synthesis. For example, the Advanced Chemtech Model 396 Multiple Peptide Synthesizer and an Applied Biosystems Model 432A Peptide synthesizer are suitable. There are commercial companies that make custom synthetic peptides to order, e.g., Abbiotec, Abgent, AnaSpec Global Peptide Services, LLC. INVITROGEN™ and rPeptide, LLC.

Alternatively, the two interacting proteins: receptor and/or ligand polypeptide can be made and purified as recombinant molecules by molecular methods that are well known in the art. For example, recombinant proteins can be expressed in bacteria, mammal, insect, yeast, or plant cells. In some embodiments, the polypeptides and functional fragments thereof used in the methods described herein are preferably recombinant proteins.

Conventional polymerase chain reaction (PCR) cloning techniques can be used to clone a nucleic acid encoding a given receptor and/or ligand polypeptide or functional fragment thereof, using the mRNA sequence coding for the intact full length polypeptide as the template for PCR cloning. In addition, one skilled in the art will be able to use PCR cloning techniques for synthesizing a chimeric nucleic acid encoding a chimeric polypeptide comprising a receptor and a corresponding ligand for that receptor or functional fragments of the receptor/ligand such that the receptor and ligand are arranged in tandem and in sufficiently close proximity to each other to facilitate their interaction with each other. Alternatively, the sense and anti-sense strand of the coding nucleic acid can be made synthetically and then annealed together to form a double-stranded coding nucleic acid.

In some embodiments, the nucleic acid sequences encoding the receptor, ligand polypeptide, functional fragments thereof, or the chimeric receptor-ligand polypeptide comprise sequences of extraneous amino acid residues that are located at the termini of the encoded subject polypeptide. The extraneous amino acid residues at the termini facilitate coupling or linking the subject polypeptide to tethering molecules to solid surfaces, e.g., dsDNA. Examples of extraneous amino acid residues include but are not limited to glycine, proline, serine, threonine, lysine and cysteine.

In other embodiments, the chimeric nucleic acid sequence can form part of a hybrid gene encoding additional polypeptide sequences, for example, sequences that function as a marker or reporter. Examples of marker or reporter genes include—lactamase, chloramphenicol acetyltransferase (CAT), adenosine deaminase (ADA), aminoglycoside phosphotransferase (neor, G418r), dihydrofolate reductase (DHFR), hygromycin-B-phosphotransferase (HPH), thymidine kinase (TK), lacZ (encoding-galactosidase), green fluorescent protein (GFP), and xanthine guanine phosphoribosyltransferase (XGPRT). As with many of the standard procedures associated with the practice of the methods described herein, skilled artisans will be aware of additional useful reagents, for example, of additional sequences that can serve the function of a marker, a reporter or facilitate purification of the polypeptide. Generally, the chimeric polypeptide will include a first portion and a second portion; the first portion being the receptor portion, for example, a portion of the VWF A1 domain amino acid sequence and the second portion being the ligand, for example, the GP1bα.

A chimeric nucleic acid sequence that form part of a hybrid gene encoding additional polypeptide sequences can be expressed with protease cleavage sites. Protease cleavage sites can be designed and included between the nucleic acid sequences to facilitate liberation of subject polypeptide from the non-subject peptide/polypeptide if so desired. For example, non-subject peptide/polypeptides include purification tags, e.g., his-tag, GST and thioredoxin expression leader polypeptides, and reporter polypeptides. Examples of protease cleavage sites include but are not limited to those of enterokinase, chymotrypsin, and thrombin.

The following are exemplary templates for use with PCR cloning in the practice of the methods described herein. The choice of PCR templates is dependent on the choice of receptor-ligand pair that is being studied.

In the embodiment where the receptor-ligand pair is a WVF A1 domain and a GP1bα subunit, the template mRNAs for PCR cloning of a DNA encoding an A1 domain and a GP1bα can be the Homo sapiens glycoprotein Ib (platelet), alpha polypeptide (GP1BA) mRNA GENBANK™ Accession No. NM_{—}000173.4; the von Willebrand factor A1 domain isoform 1 precursor mRNA GENBANK™ Accession No.NM_{—}022834.4; and the von Willebrand factor A1 domain isoform 2 precursor mRNA GENBANK™ Accession No. NM_{—}199121.2.

In the embodiment where the receptor-ligand pair is an α4b7 integrin and a madcam-1, the template mRNAs for PCR cloning of a DNA encoding an a4b7 integrin and a madcam-1 can be the Homo sapiens integrin alpha L isoform b precursor GENBANK™ Accession No. NM_{—}001114380.1; the integrin alpha L isoform a precursor GENBANK™ Accession No. NM_{—}002209.2; and the intercellular adhesion molecule 1 (ICAM-1) precursor GENBANK™ Accession No. NM_{—}000201.2.

In the embodiment where the receptor-ligand pair is an αL integrin I domain and an ICAM-1(D1+D2), the template mRNAs for PCR cloning of the DNAs encoding an αL integrin I domain and an ICAM-1(D1+D2) can be the mRNA of the integrin alpha L isoform a precursor GENBANK™ Accession No. NM_{—}002209.2 and the mRNA of the Homo sapiens intercellular adhesion molecule 1 precursor (ICAM-1) GENBANK™ Accession No. NM_{—}000201.2.

In the embodiment where the receptor-ligand pair is the αL integrin I domain and ICAM-3(D1), the template mRNAs for PCR cloning of the DNAs encoding an αL integrin I domain and an ICAM-3(D1) can be the mRNA of the integrin alpha L isoform a precursor GENBANK™ Accession No. NM_{—}002209.2 and the mRNA of the Homo sapiens intercellular adhesion molecule 3 precursor (ICAM-3) GENBANK™ Accession No. NM_{—}002162.3. The I domain encompasses amino acid residues 145-324 of the 1145 amino acid long mature αL integrin subunit protein (amino acid residues 26-1170 of GenBank Accession No. NP_{—}002200).

In the embodiment where the receptor-ligand pair is a fimH pilin+lectin domain and a N-linked carbohydrates, the template mRNA for PCR cloning the DNA encoding a fimH pilin+lectin domain can be the Escherichia coli strain J96 type 1 fimbrial adhesin precursor (fimH) gene, GENBANK™ Accession No. AY914173.

PCR amplified coding nucleic acids or annealed sense and anti-sense nucleic acid with 3′A overhang can be cloned into a vector using the TOPO® cloning method in INVITROGEN™ topoisomerase-assisted TA vectors such as pCR®-TOPO, pCR®-Blunt II-TOPO, pENTR/D-TOPO®, and pENTR/SD/D-TOPO®. Both pENTR/D-TOPO®, and pENTR/SD/D-TOPO® are directional TOPO entry vectors which allow the cloning of the DNA sequence in the 5′→3′ orientation into a Gateway® expression vector. Directional cloning in the 5′→3′ orientation facilitate the unidirectional insertion of the DNA sequence into a protein expression vector such that the promoter is upstream of the 5′ ATG start codon of the nucleic acid, thus enabling promoter-driven protein expression. The recombinant vector carrying a polypeptide coding nucleic acid can be transfected into and propagated in a general cloning E. coli cells such as XL1B1ue, SURE (STRATAGENE®) and TOP-10 cells (INVITROGEN™). Ideally, restriction enzyme digestion recognition sites should be designed at the ends of the sense and anti-sense strand to facilitate ligation into a cloning vector or other vectors. Alternatively, a 3′A overhang can be include for the purpose of TA-cloning that is well known in the art. Such coding nucleic acids with 3′A overhangs can be easily ligated into the INVITROGEN™ topoisomerase-assisted TA vectors such as pCR®-TOPO, pCR®-Blunt II-TOPO, pENTR/D-TOPO®, and pENTR/SD/D-TOPO®. The coding nucleic acid can be cloned into a general purpose cloning vector such as pUC19, pBR322, pBluescript vectors (STRATAGENE Inc.) or pCR TOPO® from INVITROGEN™ Inc. The resultant recombinant vector carrying the nucleic acid encoding a peptide can then be subcloned into protein expression vectors or viral vectors for the synthesis of recombinant proteins in a variety of protein expression systems using host cells selected from the group consisting of mammalian cell lines, insect cell lines, yeast, bacteria, and plant cells.

In one preferred embodiment, the recombinant proteins are made in the type of host cells that are closest to the native protein. For example, if the receptor-ligand pair are bacterial proteins, then it is preferred that the recombinant proteins are expressed in a prokaryotic expression system, e. g. in E. coli or Pichia; if the receptor-ligand pair are yeast proteins, then it is preferred that the recombinant proteins are expressed in a yeast expression system, e. g. Saccharomyces cerevisia; if the receptor-ligand pair are mammalian proteins, then preferably the proteins are expressed in eukaryotic cells. In one embodiment, the nucleic acids are operationally linked to a promoter.

Different expression vectors comprising a nucleic acid that encodes a receptor, ligand, and functional fragments thereof as described herein for the expression and purification of the recombinant protein produced from a heterologous protein expression system can be made. Heterologous protein expression systems that use host cells selected from, e. g., mammalian, insect, yeast, bacterial, or plant cells are well known to one skilled in the art. The expression vector should have the necessary 5′ upstream and 3′ downstream regulatory elements such as promoter sequences, ribosome recognition and binding TATA box, and 3′ UTR AAUAAA transcription termination sequence for efficient gene transcription and translation in its respective host cell. The regulatory elements referred to above include, but are not limited to, inducible and non-inducible promoters, enhancers, operators and other elements, which are known to those skilled in the art, and which drive or otherwise regulate gene expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast-mating factors. The expression vector can have additional sequence such as 6×-histidine (SEQ ID NO: 3), V5, thioredoxin, glutathione-S-transferase, c-Myc, VSV-G, HSV, FLAG, maltose binding peptide, metal-binding peptide, HA and “secretion” signals (Honeybee melittin, α-factor, PHO, Bip), which are incorporated into the expressed recombinant peptide. In addition, there can be enzyme digestion sites incorporated after these sequences to facilitate enzymatic removal of additional sequence after they are not needed. These additional sequences are useful for the detection of peptide expression, for protein purification by affinity chromatography, enhanced solubility of the recombinant protein in the host cytoplasm, for better protein expression especially for small peptides and/or for secreting the expressed recombinant protein out into the culture media, into the periplasm of the prokaryote bacteria, or to the spheroplast of yeast cells. The expression of recombinant peptide can be constitutive in the host cells or it can be induced, e.g., with copper sulfate, sugars such as galactose, methanol, methylamine, thiamine, tetracycline, infection with baculovirus, and (isopropyl-beta-D-thiogalactopyranoside) IPTG, a stable synthetic analog of lactose, depending on the host and vector system chosen.

Recombinant receptor, ligand, and functional fragments can be expressed in a variety of expression host cells e. g., bacteria, such as E. coli, yeast, mammalian, insect, and plant cells such as Chlamydomonas, or even from cell-free expression systems. From a cloning vector, the nucleic acid can be subcloned into a recombinant expression vector that is appropriate for the expression of the peptide in mammalian, insect, yeast, bacterial, or plant cells or a cell-free expression system such as a rabbit reticulocyte expression system. Subcloning can be achieved by PCR cloning, restriction digestion followed by ligation, or recombination reaction such as those of the lambda phage-based site-specific recombination using the GATEWAY® LR and BP CLONASE™ enzyme mixtures. Subcloning should be unidirectional such that the 5′ ATG start codon of the nucleic acid is downstream of the promoter in the expression vector. Alternatively, when the coding nucleic acid is cloned into pENTR/D-TOPO®, pENTR/SD/D-TOPO® (directional entry vectors), or any of the INVITROGEN™'s GATEWAY® Technology pENTR (entry) vectors, the coding nucleic acid can be transferred into the various GATEWAY® expression vectors (destination) for protein expression in mammalian cells, E. coli, insects and yeast respectively in one single recombination reaction. Some of the GATEWAY® destination vectors are designed for the constructions of baculovirus, adenovirus, adeno-associated virus (AAV), retrovirus, and lentiviruses, which upon infecting their respective host cells, permit heterologous expression of the peptide in the host cells. Transferring a gene into a destination vector is accomplished in just two steps according to manufacturer's instructions. There are GATEWAY® expression vectors for protein expression in E. coli, insect cells, mammalian cells, and yeast. Following transformation and selection in E. coli, the expression vector is ready to be used for expression in the appropriate host.

Examples of other expression vectors and host cells are the pET vectors (NOVAGEN), pGEX vectors (Amersham Pharmacia) and pMAL vectors (New England labs. Inc.) for protein expression in E. coli host cells such as BL21, BL21(DE3) and AD494(DE3)pLysS, Rosetta (DE3), and Origami(DE3) (NOVAGEN); the strong CMV promoter-based pcDNA3.1 (INVITROGEN) and pClneo vectors (PROMEGA) for expression in mammalian cell lines such as CHO, COS, HEK-293, Jurkat, and MCF-7; replication incompetent adenoviral vector vectors pAdeno X, pAd5F35, pLP-Adeno-X-CMV (CLONTECH), pAd/CMV/V5-DEST, pAd-DEST vector (INVITROGEN) for adenovirus-mediated gene transfer and expression in mammalian cells; pLNCX2, pLXSN, and pLAPSN retrovirus vectors for use with the RETRO-X™ system from Clontech for retroviral-mediated gene transfer and expression in mammalian cells; pLenti4/V5-DEST™, pLenti6/V5-DEST™, and pLenti6.2N5-GW/lacZ (INVITROGEN™) for lentivirus-mediated gene transfer and expression in mammalian cells; adenovirus-associated virus expression vectors such as pAAV-MCS, pAAV-IRES-hrGFP, and pAAV-RC vector (STRATAGENE®) for adeno-associated virus-mediated gene transfer and expression in mammalian cells; BACpak6 baculovirus (CLONTECH) and pFastBac™ HT (INVITROGEN™) for the expression in Spodopera frugiperda 9 (Sf9) and Sf11 insect cell lines; pMT/BiP/V5-His (INVITROGEN™) for the expression in Drosophila Schneider S2 cells; Pichia expression vectors pPICZα, pPICZ, pFLDα and pFLD (INVITROGEN™) for expression in Pichia pastoris and vectors pMETα and pMET for expression in P. methanolica; pYES2/GS and pYD1 (INVITROGEN™) vectors for expression in yeast Saccharomyces cerevisiae. Recent advances in the large scale expression heterologous proteins in Chlamydomonas reinhardtii are described by Griesbeck C. et al. 2006 Mol. Biotechnol. 34:213-33 and Fuhrmann M. 2004, Methods Mol Med. 94:191-5. Foreign heterologous coding sequences are inserted into the genome of the nucleus, chloroplast and mitochondria by homologous recombination. The chloroplast expression vector p64 carrying the versatile chloroplast selectable marker aminoglycoside adenyl transferase (aadA), which confers resistance to spectinomycin or streptomycin, can be used to express foreign protein in the chloroplast. The biolistic gene gun method can be used to introduce the vector in the algae. Upon its entry into chloroplasts, the foreign DNA is released from the gene gun particles and integrates into the chloroplast genome through homologous recombination.

The expression systems that can be used for synthesizing the recombinant receptor, ligand and functional fragments thereof include, but are not limited to, microorganisms such as bacteria (for example, E. coli and B. subtilis) transformed with recombinant bacteriophage DNA, plasmid DNA, or cosmid DNA expression vectors containing the nucleic acid molecules encoding the recombinant receptor, ligand and functional fragments thereof; yeast (for example, Saccharomyces and Pichia) transformed with recombinant yeast expression vectors containing the nucleic acid molecules encoding recombinant receptor, ligand and functional fragments thereof; insect cell systems infected with recombinant virus expression vectors (for example, baculovirus) containing the nucleic acid molecules recombinant receptor, ligand and functional fragments thereof; plant cell systems infected with recombinant virus expression vectors (for example, cauliflower mosaic virus (CaMV) and tobacco mosaic virus (TMV)) or transformed with recombinant plasmid expression vectors (for example, Ti plasmid) containing nucleotide sequences encoding the recombinant receptor, ligand and functional fragments thereof; or mammalian cell systems (for example, COS, CHO, BHK, 293, VERO, HeLa, MDCK, W138, and NIH 3T3 cells) harbouring recombinant expression constructs containing promoters derived from the genome of mammalian cells (for example, the metallothionein promoter) or from mammalian viruses (for example, the adenovirus late promoter and the vaccinia virus 7.5K promoter).

In bacterial systems, a number of expression vectors can be advantageously selected depending upon the method of purification of the gene product being expressed. Such vectors include, but are not limited to, the E. coli expression vector pUR278 (Ruther et al., EMBO J. 2:1791, 1983), in which the coding sequence of the subject polypeptide can be ligated individually into the vector in frame with the lacZ coding region so that a fusion protein is produced; pIN vectors (Inouye and Inouye, Nucleic Acids Res. 13:3101-3109, 1985; Van Heeke and Schuster, J. Biol. Chem. 264:5503-5509, 1989); and the like. pGEX vectors can also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhidrosis virus (AcNPV) can be used as a vector to express foreign genes. The virus grows in Spodoptera frugiperda cells. The coding sequence of the subject polypeptide can be cloned individually into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (e.g., see Smith et al., J. Virol. 46:584, 1983; Smith, U.S. Pat. No. 4,215,051).

In mammalian host cells, a number of viral-based expression systems can be utilized. In cases where an adenovirus is used as an expression vector, the nucleic acid molecule of the invention can be ligated to an adenovirus transcription/translation control complex, for example, the late promoter and tripartite leader sequence. This chimeric nucleic acid sequence can then be inserted in the adenovirus genome by in vitro or in vivo recombination. Insertion in a non-essential region of the viral genome (for example, region E1 or E3) will result in a recombinant virus that is viable and capable of expressing a recombinant receptor, ligand and functional fragments thereof in infected hosts (for example, see Logan and Shenk, Proc. Natl. Acad. Sci. USA 81:3655-3659, 1984). Specific initiation signals can also be required for efficient translation of inserted nucleic acid molecules. These signals include the ATG initiation codon and adjacent sequences. In cases where an entire gene or cDNA, including its own initiation codon and adjacent sequences, is inserted into the appropriate expression vector, no additional translational control signals is needed. However, in cases where only a portion of the coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol 153:516-544, 1987).

In addition, a host cell strain can be chosen, which modulates the expression of the inserted sequences, or modifies and processes the gene product in the specific fashion desired. Such modifications (for example, glycosylation) and processing (for example, cleavage) of protein products can be important for the function of the protein. Different host cells have characteristic and specific mechanisms for the posttranslational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. The mammalian cell types listed above are among those that could serve as suitable host cells.

A number of selection systems can be used. For example, the herpes simplex virus thymidine kinase (Wigler, et al., Cell 11: 223, 1977), hypoxanthine-guanine phosphoribosyltransferase (Szybalska and Szybalski, Proc. Natl. Acad. Sci. USA 48:2026, 1962), and adenine phosphoribosyltransferase (Lowy, et al., Cell 22:817, 1980) genes can be employed in tk-, hgprt- or aprt-cells, respectively. Also, anti-metabolite resistance can be used as the basis of selection for the following genes: dhfr, which confers resistance to methotrexate (Wigler et al., Proc. Natl. Aced. Sci. USA 77:3567, 1980; O\'Hare et al., Proc. Natl. Acad. Sci. USA 78:1527, 1981); gpt, which confers resistance to mycophenolic acid (Mulligan and Berg, Proc. Natl. Acad. Sci. USA 78:2072, 1981); neo, which confers resistance to the aminoglycoside G-418 (Colberre-Garapin et al., J. Mol. Biol. 150:1, 1981); and hygro, which confers resistance to hygromycin (Santerre et al., Gene 30:147, 1984).

Cell-free expression systems are also contemplated. Cell-free expression systems offer several advantages over traditional cell-based expression methods, including the easy modification of reaction conditions to favor protein folding, decreased sensitivity to product toxicity and suitability for high-throughput strategies such as rapid expression screening or large amount protein production because of reduced reaction volumes and process time. The cell-free expression system can use plasmid or linear DNA. Moreover, improvements in translation efficiency have resulted in yields that exceed a milligram of protein per milliliter of reaction mix. An example of a cell-free translation system capable of producing proteins in high yield is described by Spirin AS. et al., Science 242:1162 (1988). The method uses a continuous flow design of the feeding buffer which contains amino acids, adenosine triphosphate (ATP), and guanosine triphosphate (GTP) throughout the reaction mixture and a continuous removal of the translated polypeptide product. The system uses E. coli lysate to provide the cell-free continuous feeding buffer. This continuous flow system is compatible with both prokaryotic and eukaryotic expression vectors. As an example, large scale cell-free production of the integral membrane protein EmrE multidrug transporter is described by Chang G. el. al., Science 310:1950-3 (2005).

Other commercially available cell-free expression systems include the EXPRESSWAY™ Cell-Free Expression Systems (INVITROGEN™) which utilize an E. coli-based in-vitro system for efficient, coupled transcription and translation reactions to produce up to milligram quantities of active recombinant protein in a tube reaction format; the Rapid Translation System (RTS) (Roche Applied Science) which also uses an E. coli-based in-vitro system; and the TNT Coupled Reticulocyte Lysate Systems (PROMEGA) which uses a rabbit reticulocyte-based in-vitro system.

Recombinant protein expression in different host cells can be constitutive or inducible with inducers such as copper sulfate, or sugars such as galactose, methanol, methylamine, thiamine, tetracycline, or IPTG. After the protein is expressed in the host cells, the host cells are lysed to liberate the expressed protein for purification. Methods of lysing the various host cells are featured in “Sample Preparation-Tools for Protein Research” EMD Bioscience and in the Current Protocols in Protein Sciences (CPPS). A preferred purification method is affinity chromatography such as ion-metal affinity chromatograph using nickel, cobalt, or zinc affinity resins for histidine-tagged peptide. Methods of purifying histidine-tagged recombinant proteins are described by CLONTECH using their TALON® cobalt resin and by NOVAGEN in their pET system manual, 10th edition. For example, a system described by Janknecht et al. allows for the ready purification of non-denatured fusion proteins expressed in human cell lines (Proc. Natl. Acad. Sci. USA 88: 8972-8976, 1991). In this system, the gene of interest is subcloned into a vaccinia recombination plasmid such that the gene's open reading frame is translationally fused to an aminoterminal tag consisting of six histidine residues (SEQ ID NO: 3). Extracts from cells infected with recombinant vaccinia virus are loaded onto Ni²⁺ nitriloacetic acid-agarose columns and histidine-tagged proteins are selectively eluted with imidazole-containing buffers.

Another preferred purification strategy is by immuno-affinity chromatography, for example, anti-Myc antibody conjugated resin can be used to affinity purify Myc-tagged peptide. Enzymatic digestion with serine proteases such as thrombin and enterokinase cleave and release the peptide from the histidine or Myc tag, releasing the recombinant peptide from the affinity resin while the histidine-tags and Myc-tags are left attached to the affinity resin. Alternatively, any fusion or chimeric protein can be readily purified by utilizing an antibody specific for the fusion protein being expressed.

### Linkers for Linking Two Polypeptides and for Tethering Polypeptides to Surfaces

In certain embodiments, the receptors, ligands, polypeptides, binding entities or functional fragments thereof described herein of a receptor-ligand pair or a bound pair can be linked together by covalent attachment to at least one linker moiety wherein the linking is in tandem and in close proximity sufficient for the receptor and ligand interaction. In one embodiment, the linker moiety is a peptide, such as a spacer linker peptide. In another embodiment, the linker moiety is not a peptide, such as a chemical linker (e.g., PEG) described herein.

In certain embodiments, the receptors, ligands or functional fragments thereof described herein of a receptor-ligand pair can be tethered to solid surfaces. For example, nanometer sized microsphere beads for use with an optical tweezer or silanized glass slide or cantilever when used with an AFM. Commonly, the microsphere is about a 500 nm polystyrene or silica microsphere, but many other possibilities exist, such as: 2.8 micron polystyrene microspheres with embedded maghemite superparamagnetic nanocrystals; small gold nanoparticles; quantum dots. A skilled artisan can use any methods known in the art for tethering polypeptides to surfaces for the practice of the methods described herein, e. g. via dsDNA. The dsDNA can have thiol derivative nucleoside for the purpose of covalently linking with the polypeptide. In the embodiment where dsDNA is used to tether the polypeptide to a surface, one end of the dsDNA is attached to the surface while the other end is linked to the polypeptide.

In some embodiments, single cysteine residues are introduced into the termini of the receptors, ligands or functional fragments thereof described herein to facilitate disulfide covalent bond with linker moieties that have thiol groups such as thiol derivative nucleoside containing dsDNA. Examples are shown in FIG. 4A and FIG. 13.

In some embodiments, several extraneous amino acid residues are introduced to the termini of the polypeptides described herein. The number of amino acid residues can range from 1-10. In some embodiments, one, two, three, four, five, six, seven, eight, nine or ten extraneous amino acid residues are introduced to the termini of the polypeptides described herein. In one embodiment, the extraneous amino acid residues introduced to the termini of the polypeptides described herein comprise at least one cysteine (see FIG. 4A).

In some aspects, the receptors, ligands or functional fragments thereof described herein of a receptor-ligand pair can be linked together physically in tandem by a spacer linker peptide. A “spacer linker peptide” is a relatively short (e.g., about 1-20, 1-40, 2-50, 2-100, 2-150, 5-50, 5-100, 5-150, 5-200, 20-50, 20-100, 20-150, 20-200, 1-200 amino acids) sequence of amino acids that is not part of the subject polypeptide under study described herein. A spacer linker peptide is attached on its amino-terminal end to one polypeptide or polypeptide domain and on its carboxyl-terminal end to the other polypeptide or polypeptide domain. Examples of useful spacer linker peptides include, but are not limited to, glycine polymers ((G)n) including glycine-serine and glycine-alanine polymers (e.g., a (Gly4Ser)n repeat where n=1-8 (SEQ ID NO: 4), preferably, n=3, 4, 5, or 6). The subject polypeptides described herein can also be joined by chemical bond linkages, such as linkages by disulfide bonds or by chemical bridges. The receptor, ligands or functional fragments thereof described herein of a receptor-ligand pair can also be linked together using non-peptide cross-linkers (Pierce 2003-2004 Applications Handbook and Catalog, Chapter 6) or other scaffolds such as HPMA, polydextran, polysaccharides, ethylene-glycol, poly-ethylene-glycol, glycerol, sugars, and sugar alcohols (e.g., sorbitol, mannitol). Non-peptide linkers can also be used to tether a subject polypeptide to a solid surface.

In one embodiment, the linker moiety is a peptide linker In one embodiment, the peptide linker has the sequence of: TGGPTIKPPKPPKPAPNLLGGPDKTHTKPPKPAPELLGGPGTG (SEQ. ID. NO: 1), which is modified from the hinge regions of murine IgG2a and human IgG1, with cysteine residues either removed or substituted with proline.

In another embodiment, the peptide linker has the sequence of GATPQDLNTML(SEQ. ID. NO: 5), corresponding to amino acids 46-56 of the human immunodeficiency virus type 1 (HIV-1) capsid protein p24.

In another embodiment, the linker moiety is a non-peptide cross-linker The linker moiety can be a C₁₋₁₂ linking moiety optionally terminated with one or two —NH— linkages and optionally substituted at one or more available carbon atoms with a lower alkyl substituent. In some embodiments, the linker comprises —NH—R—NH— wherein R is a lower (C1-6) alkylene substituted with a functional group, such as a carboxyl group or an amino group, that enables binding to another molecular moiety (e.g., as may be present on the surface of a solid support during peptide synthesis or to a pharmacokinetic-modifying agent such as PEG). In certain embodiments, the linker has a lysine residue. In certain other embodiments, the linker bridges the C-termini of two polypeptides, by simultaneous attachment to the C-terminal amino acid of each polypeptide. In other embodiments, the linker bridges the subject polypeptides by attaching to the side chains of amino acids not at the C-termini. When the linker attaches to a side chain of an amino acid not at the C-termini of the polypeptides, the side chain preferably contains an amine, such as those found in lysine, and the linker contains two or more carboxy groups capable of forming an amide bond with the polypeptides. In a preferred embodiment, the linker does not attach to the functional fragment of the receptor or ligand when the receptor and ligand are two separate and independent polypeptides and the linker is used to couple these two polypeptides together.

In an optional embodiment, polyethylene glycol (PEG) serves as a linker that joins the receptor or functional fragments thereof to the ligand or functional fragments thereof. For example, a single PEG moiety containing two reactive functional groups can be simultaneously attached to the N-termini of both polypeptide chains.

In another embodiment, a linker moiety can comprise a molecule containing two carboxylic acids and optionally substituted at one or more available atoms with an additional functional group such as an amine capable of being bound to one or more PEG molecules. Such a molecule can be depicted as: —CO—(CH₂)n-uX—(CH₂)m-CO— where n is an integer between zero and 10, m is an integer between one and 10, X is selected from O, S, N(CH₂)pNR1, NCO(CH₂)pNR1, and CHNR1, R1 is selected from H, Boc (tert-butyloxycarbonyl), Cbz, and p is an integer between 1 and 10. In certain embodiments, one amino group of each of the polypeptides forms an amide bond with the linker In certain other embodiments, the amino group of the polypeptide bound to the linker is the epsilon amine of a lysine residue or the alpha amine of the N-terminal residue, or an amino group of an optional spacer molecule. In another embodiment, a spacer can be used in addition to a linker molecule for separating moieties as desired. In some embodiments, both n and m are one, X is NCO(CH₂)pNR1, p is two, and R1 is Boc. Optionally, the Boc group can be removed to liberate a reactive amine group capable of forming a covalent bond with a suitably activated PEG species such as mPEG-SPA-NHS or mPEG-NPC (Nektar Therapeutics, San Carlos Calif.). Optionally, the linker contains more than one reactive amine capable of being derivatized with a suitably activated PEG species. Optionally, the linker contains one or more reactive amines capable of being derivatized with a suitably activated pharmacokinetic (PK) modifying agent such as a fatty acid, a homing peptide, a transport agent, a cell-penetrating agent, an organ-targeting agent, or a chelating agent. PEG can be purchased with array of various functional terminal groups. One of the most common functionalities is a bis-amino termination of PEG molecule. Tethering this PEG molecule to aminated solid surfaces can be achieved with homobifunctional cross-linkers such as PDITC (1, 4-phenylenediisothiocyanate). Methods of tethering polypeptides with PEG crosslinking agents are known in the art, e.g., dimaleimido-PEG cross-linking agent as described by D. J. Cipriano and S. D. Dunn in Proteins: Structure, Function, and Bioinformatics, 2008, 73:458-467. In tethering the polypeptides to surfaces, the goal is to attach one terminus of the PEG linker to the surface (e.g. beads, slides, cantilever etc) and to functionalize the other terminus of the PEG linker to become amino- or sulfhydryl-reactive. Amino-reactivity can be achieved by reacting again with PDITC and sulfhydryl-reactivity can be obtained by using MaleimidoBenzoyl-N-HydroxySuccinimidyl ester (MBS). Polypeptides with containing sulfhydryl (—SH) groups can be then react with the surface-attached activated PEG linkers.

In some embodiments, the linker moiety has the following structure: —NH—(CH2)α—[O—(CH2)β]γ—Oδ—(CH2)ε—Y— where α, β, γ, δ, and ε are each integers whose values are independently selected. In some embodiments, α, β, and ε are each integers whose values are independently selected between one and about six, δ is zero or one, γ is an integer selected between zero and about ten, except that when γ is greater than one, β is two, and Y is selected from NH or CO. In some embodiments, α, β, and ε are each equal to two, both γ and δ are equal to 1, and Y is NH. In another embodiment, γ and δ are zero, α and ε together equal five, and Y is CO.

By far the two most popular strategies for tethering molecules to surfaces are the biotin-streptavidin and digoxenin (dig)/anti-digoxenine (anti-dig) antibodies.

The polypeptides can be linked using the biotin/streptavidin system. Biotinylated analogs of subject polypeptides can be synthesized by standard techniques known to those skilled in the art. For example, the polypeptides can be C-terminally biotinylated. These biotinylated polypeptides are then streptavidin coated microspheres or streptavidin coated glass slides. In the embodiments where dsDNA is used to tether the polypeptide to a surface, one end of the dsDNA is attached to the surface while the other end is linked to the polypeptide. On common method is to label the dsDNA with digoxigenin (dig) or biotin on the other end and have at least one thiol derivative nucleoside at the opposite end. The thiol group reacts with cysteine at the terminus of the polypeptide to form disulpide bonds with the polypeptide and thus linking the dsDNA to the subject polypeptide (See FIG. 9). The surface of the glass slide or microsphere bead can be coated with a polyclonal antibody against dig (anti-dig), coated with streptavidin which binds to biotin with high affinity or coated anti-biotin antibodies (e.g., goat anti-biotin IgG from Kirkegaard & Perry Laboratories, Inc. (Washington, D.C.). The binding of dig to anti-dig, biotin-streptavidin, or biotin to anti-biotin links the dsDNA or other handles to the surface of a glass slide or a microsphere. These make the following tethering schemes:
solid surface—anti-dig/dig—dsDNA—S—S bond—subject polypeptide;
solid surface—streptavidin-biotin—dsDNA—S—S bond—subject polypeptide; and
solid surface—anti-biotin-biotin—dsDNA—S—S bond—subject polypeptide;

When PEG500 is used in place of a dsDNA handle, the following tethering scheme can be obtained:
solid surface—anti-biotin-biotin—PEG500—S—S bond—subject polypeptide.

Other methods for tethering the end of the dsDNA or polypeptides directly to surfaces include but are not limited to anti-HA/HA-RNA polymerase-DNA; gold/thiol-DNA; anti-histidine/histidine-Protein-DNA; and anti-fluorescein/fluorescein-DNA systems.

In some embodiments, the surfaces are silanized prior to attaching the polypeptide. This is a method of amination of silanol-containing surfaces and is commonly used in microscopy such as an AFM described herein. Many such amination methods are known in the art. One skilled in the art would be able to select an amination method appropriate for the “stretching out” approach adopted (i. e. optical tweezer or AFM) for the particular polypeptide being studied. For example, reduced frequency of force artifacts and small-range (up to micron scale) constant force features are desirable when using AFM. One common method of amination of silanol-containing surfaces is silanization with 3-aminopropyltriethoxysilane (APTES). An alternative method of amination of silanol-containing surfaces is amination with ethanolamine. Other methods of tethering polypeptides to surfaces for AFM studies can be found in Peter Hinterdorfer et al., Chem. Phys. Chem. 2003, 4:1367-1371; Biochem. 97: 1191-1197, 2006; and Ariel Fernandez et al. J. Phys. Chem. B 2007, 111:13987-13992 and these references are incorporated herein by reference in their entirety.

### Libraries for Screening

The agents that can be screened for modulation activity of a receptor-ligand interaction include, but are not limited to, biomolecules, including, but not limited to, amino acids, peptides, polypeptides, peptiomimetics, nucleotides, nucleic acids (including DNA, cDNA, RNA, antisense RNA and any double- or single-stranded forms of nucleic acids and derivatives and structural analogs thereof), polynucleotides, saccharides, fatty acids, steroids, carbohydrates, lipids, lipoproteins and glycoproteins. Such biomolecules can be substantially purified, or can be present in a mixture, such as a cell extract or supernate. Agents further include synthetic or natural chemical compounds, such as simple or complex organic molecules, metal-containing compounds and inorganic ions. Also included are pharmacological compounds, which optionally can be subjected to directed or random chemical modifications, such as acylation, alkylation, esterification, amidation, etc., to produce structural analogs.

In some embodiments, the agent is an agent of interest including known and unknown compounds that encompass numerous chemical classes, primarily organic molecules, which may include organometallic molecules, inorganic molecules, genetic sequences, etc. An important aspect of the methods described herein is the evaluation of candidate drugs, including toxicity testing; and the like that can modulate proteins that have been implicated in certain diseases, disorder and/or medical pathology. Candidate agents also include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Also included as agents are pharmacologically active drugs, genetically active molecules, etc. Compounds of interest include, for example, chemotherapeutic agents, hormones or hormone antagonists, growth factors or recombinant growth factors and fragments and variants thereof. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

The agents include all of the classes of molecules described above, and can further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g., lysates prepared from crops, tissue samples, etc.; manufacturing samples, e.g., time course during preparation of pharmaceuticals; as well as libraries of compounds prepared for analysis; and the like. Samples of interest include compounds being assessed for potential therapeutic value, i.e. drug candidates.

Agents such as chemical compounds that are useful for the screening methods described herein, including candidate agents or candidate drugs, can be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents can be subjected to directed or random chemical modifications, such as acylation, alkylation, esterification, amidification, etc. to produce structural analogs.

In addition, compound libraries are also available from commercial sources. For example, libraries from Vitas-M Lab and Biomol International, Inc. A comprehensive list of compound libraries can be found at the World Wide Website at

broad.harvard.edu/chembio/platform/screening/compound_libraries/index.htm. Other chemical compound libraries such as those from of 10,000 compounds and 86,000 compounds from NIH Roadmap, Molecular Libraries Screening Centers Network (MLSCN) can be screened. A chemical library or compound library is a collection of stored chemicals usually used ultimately in high-throughput screening or industrial manufacture. The chemical library can consist in simple terms of a series of stored chemicals. Each chemical has associated information stored in some kind of database with information such as the chemical structure, purity, quantity, and physiochemical characteristics of the compound.

In some embodiments, agents include the known agonist, antagonist and inhibitors of the particular receptor-ligand interaction that is studied; e.g., ristocetin and botrocetin are both agonists of the VWF A1 domain/GP1bα interaction, as they strengthen the interaction (see FIG. 5).

The present invention can be defined in any of the following numbered paragraphs:
1. A method of screening for a modulator of an interaction between a receptor and a ligand pair, the method comprising: (a) contacting a ligand-bound-receptor protein with an agent; (b) extending the ligand-bound-receptor protein; (c) monitoring a signal that represents the protein existing in either a ligand-bound state or in a ligand-unbound state and the transition between the two states; and (d) comparing the signal with a reference signal wherein a deviation from the reference indicate that the agent is a modulator.
2. The method of paragraph 1, wherein the reference is that of the ligand-bound-receptor protein in the absence of a modulator.
3. The method of paragraph 1 or 2, wherein the extending of the ligand-bound-receptor protein occurs with an optical tweezer or an atomic force microscope (AFM).
4. The method of any of paragraphs 1-3, wherein the extending of the ligand-bound-receptor protein occurs with a mobile focus laser light, a cantilever, or a positioner in the AFM.
5. The method of any of paragraphs 1-4, wherein the signal is a force required to dissociate the ligand receptor interaction and/or produce an increase in extension of the ligand-bound-receptor protein.
6. The method of paragraph 5, wherein a positive deviation of at least 10% from the reference indicates that the modulator is an agonist of the receptor-ligand interaction.
7. The method of paragraph 5, wherein a negative deviation of at least 10% from the reference indicates that the modulator is an antagonist of the receptor-ligand interaction.
8. The method of any of paragraphs 1-4, wherein the signal is a rate of dissociation of the ligand receptor interaction and/or a dissociation constant of the rate.
9. The method of paragraph 8, wherein a negative deviation of at least 10% from the reference indicates that the modulator is an agonist of the receptor-ligand interaction.
10. The method of paragraph 8, wherein a positive deviation of at least 10% from the reference indicates that the modulator is an antagonist of the receptor-ligand interaction.
11. The method of any of paragraphs 1-10, wherein the ligand-bound-receptor protein is a chimeric fusion protein comprising (1) a receptor or ligand-binding fragments thereof and (2) a ligand or receptor-binding fragment thereof, wherein the receptor or ligand-binding fragments thereof and the ligand or receptor-binding fragment thereof are fused together in a single polypeptide;
12. The method of any of paragraphs 1-10, wherein the ligand-bound-receptor protein is a complex of two independent polypeptides wherein one polypeptide comprises a receptor or ligand-binding fragments thereof and the other polypeptide comprises a ligand or receptor-binding fragment thereof; wherein the complexing is by way of the ligand-receptor interaction; and wherein the two polypeptides are linked by non-covalent bonds located at non-ligand binding/non receptor-binding regions of the polypeptides.
13. The method of any of paragraphs 1-10, wherein the ligand-bound-receptor protein is a complex of two independent polypeptides wherein one polypeptide comprises a receptor or ligand-binding fragments thereof and the other polypeptide comprises a ligand or receptor-binding fragment thereof; wherein the complexing is by way of the ligand-receptor interaction; and wherein the two polypeptides are linked by covalent bonds located at non-ligand binding/non receptor-binding regions of the polypeptides.
14. The method of any of paragraphs 1-13, wherein the ligand is a natural ligand of the receptor.
15. The method of any of paragraphs 1-14, wherein the ligand is an artificial ligand of the receptor.
16. The method of any of paragraphs 1-15, wherein the receptor or ligand-binding fragments thereof and the ligand or receptor-binding fragment thereof are separated by a spacer linker peptide.
17. The method of paragraph 16, wherein the spacer linker peptide has at least one amino acid residue and up to about 200 amino acid residues.
18. The method of any of paragraphs 1-17, wherein both amino and carboxyl ends of the protein are tethered to a handle for use with the optical tweezer or an AFM.
19. The method of any of paragraphs 1-18, wherein only one end of the protein is tethered to a handle for use with the optical tweezers or atomic force microscope.
20. The method of paragraph 18 or 19, wherein the handle is a double-stranded DNA.
21. The method of any of paragraphs 1-20, wherein the receptor-ligand pair is VWF A1 domain and GP1bα subunit.
22. The method of any of paragraphs 1-20, wherein the receptor-ligand pair is α4b7 integrin-madcam-1
23. The method of any of paragraphs 1-20, wherein the receptor-ligand pair is αL integrin I domain—ICAM-1(D1+D2).
24. The method of any of paragraphs 1-20, wherein the receptor-ligand pair is αL integrin I domain—ICAM-3 (D1).
25. The method of any of paragraphs 1-20, wherein the receptor-ligand pair is fimH pilin+lectin domain—N-linked carbohydrate.

Unless otherwise explained, all technical and scientific terms used herein have the same meaning as commonly understood by one of ordinary skill in the art to which this disclosure belongs. The terminology used herein is for the purpose of describing particular embodiments only, and is not intended to limit the scope of the present invention, which is defined solely by the claims. Definitions of common terms in molecular biology can be found in Robert S. Porter et al. (eds.), The Encyclopedia of Molecular Biology, published by Blackwell Science Ltd., 1994 (ISBN 0-632-02182-9); Robert A. Meyers (ed.), Molecular Biology and Biotechnology: a Comprehensive Desk Reference, published by VCH Publishers, Inc., 1995 (ISBN 1-56081-569-8); and Benjamin Lewin, Genes IX, published by Jones & Bartlett Publishing, 2007 (ISBN-13: 9780763740634); Kendrew et al. (eds.), which are all incorporated herein by reference in their entirety.

Unless otherwise stated, the methods described herein are performed using standard procedures, as described, for example in Maniatis et al., Molecular Cloning: A Laboratory Manual, Cold Spring Harbor Laboratory Press, Cold Spring Harbor, N.Y., USA (1982); Sambrook and Russel, Molecular Cloning: A Laboratory Manual (3rd ed.), Cold Spring Harbor Laboratory Press, Cold Spring Harbor, N.Y., USA (2001); Davis et al., Basic Methods in Molecular Biology, Elsevier Science Publishing, Inc., New York, USA (1986); Methods in Enzymology: Guide to Molecular Cloning Techniques Vol.152, S. L. Berger and A. R. Kimmerl Eds., Academic Press Inc., San Diego, USA (1987)); Current Protocols in Molecular Biology (CPMB) (Fred M. Ausubel, et al. ed., John Wiley and Sons, Inc.); and Current Protocols in Protein Science (CPPS) (John E. Coligan, et. al., ed., John Wiley and Sons, Inc.); which are all incorporated herein by reference in their entirety. It should be understood that the methods described herein are not limited to the particular methodology, protocols, and reagents, etc., described herein and as such may vary.

Other than in the operating examples, or where otherwise indicated, all numbers expressing quantities of ingredients or reaction conditions used herein should be understood as modified in all instances by the term “about.” The term “about” when used in connection with percentages may mean ±1%.

The singular terms “a,” “an,” and the include plural referents unless context clearly indicates otherwise. Similarly, the word or is intended to include and unless the context clearly indicates otherwise. It is further to be understood that all base sizes or amino acid sizes, and all molecular weight or molecular mass values, given for polypeptides are approximate, and are provided for description. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

This invention is further illustrated by the following example which should not be construed as limiting. The contents of all references cited throughout this application, as well as the figures and Table are incorporated herein by reference in their entirety.

### EXAMPLE

Hemostasis in the arteriolar circulation mediated by von Willebrand factor (VWF) binding to platelets is an example of an adhesive interaction that must withstand strong hydrodynamic forces acting on cells. VWF is a highly multimerized, multifunctional protein that has binding sites for platelets as well as subendothelial collagen (FIG. 2). It plays a key role in initiating hemostasis and forming arterial thrombi, especially where shear is high. It is the largest soluble protein, and is disulfide linked at both its N and C-termini in concatamers that range up to 50,000,000 Mr. Binding of the A1 domain in VWF to the glycoprotein Ibα subunit (GPIbα) on the surface of platelets (FIG. 2) mediates crosslinking of platelets to one another, initiating VWF-mediated platelet aggregation and the formation of a platelet plug for arterioles (FIG. 2). The importance of VWF is illustrated by its mutation in von Willebrand disease, a bleeding diathesis.

In order to mediate platelet aggregation, this receptor-ligand bond between VWF and G PIbα must resists substantial forces exerted on cells in vascular shear flow. To understand the effect of mechanical stress on the A1-GPIbα bond, single molecule force spectroscopy was applied to a covalently tethered receptor-ligand construct expressed in mammalian cells. This has allowed the study of dissociation/association dynamics of individual receptor-ligand bonds over many cycles, and to discover a novel specialization to resist tensile force.

Protein expression. The cDNA of human VWF A1 domain (Ile1262 to Pro1466 with pre-pro-VWF numbering), and human platelet GPIbα (His1 to Arg290) were PCR-amplified, and then used to construct the cDNAs of covalently tethered A1-GPIbα with or without additional cysteines flanking the N and C termini (FIG. 4A).The sequence of the peptide linker

(TGGPTIKPPKPPKPAPNLLGGPDKTHTKPPKPAPELLGGPGTG; SEQ. ID. NO:1) was modified from the hinge regions of murine IgG2a and human IgG1, where Cys residues were either removed or substituted with Pro. All Lys residues were followed by Pro; the Lys-Pro sequence is resistant to trypsin cleavage. The engineered cDNAs were cloned into Age I and Xho I sites of plasmid pHLsec, which encodes a Kozak sequence, a N-terminal secretion signal sequence, a vector derived ET sequence, and a C-terminal His6 tag (SEQ ID NO: 3). HEK293T cells were transiently transfected using calcium phosphate. Culture supernatants were harvested 3 days after transfection and proteins were purified using Ni-NTA affinity chromatography followed by size-exclusion chromatography in 20 mM Tris, pH 8.0, 50 mM NaCl, 0.02% NP-40, and 5 mM EDTA.

Sample preparation. Several 802-bp DNA handles were PCR-amplified using forward primers with a 5′thiol group and reverse primers with either 5′biotin or 5′digoxigenin, and activated with 2,2′-dithio-dipyridine (DTDP) as described in Cecconi et al., Eur. Biophys. J., 2008, 37:729-738. For protein-DNA coupling, 1 μM of protein (100 μL) was incubated in 0.1 mM DTT for 30 min under argon at room temperature, followed by removing DTT with 0.5 ml Zeba desalting columns (PIERCE) twice. About 0.1 μM of protein was allowed to react with 0.1 μM of each DTDP-activated DNA handle in 20 mM Tris, pH 7.5, 100 mM NaCl, 0.01% NP-40, and 1 mM EDTA under argon for 16 hours (typically 50 μL). Protein coupling to DNA handles was assayed by 4-20% native gels (FIG. 4B). Material was stored at −80° C.

Carboxyl-polystyrene beads of 2.1 and 4.3 μm diameter (Spherotech, Lake Forest, Ill.) were washed and resuspended in 0.2 mL of 50 mM 2-[N-morpholino]ethanesulfonic acid pH 5.2, 0.05% ProClin 300 (Bangs Labs, Fishers, Ind.). 1-ethyl-3-(3-dimethylaminopropyl)carbodiimide (2 mg in 10 uL of the same buffer) was added, followed after 5 min by 50 ug of 5 mg/mLstreptavidin (INVITROGEN™) or 1 mg/mL affinity-purified sheep anti-digoxigeninFab (ROCHE) in PBS. After shaking for 1 hour at room temperature, beads were washed 5 times in PBS and stored at 4° Cin PBS supplemented with 0.02% Tween 20 and 2 mM sodium azide.

FIG. 2 shows as an illustration of VWF-mediated platelet aggregation. Platelets form thrombi at sites of vascular injury to stop bleeding. Hemostasis at sites of high shear such as in arteries is dependent on VWF. VWF crosslinks platelets to one another, and also links platelets to endothelial cells and the basement membrane, to form a platelet plug.

FIG. 3C shows the contour length and the persistence length for CVT A1-GPIbα were calculated as 18.5±0.8 nm and 0.8±0.16 nm, respectively. The measured contour length agrees with the expected contour length of 18.2 nm, calculated as 1.9 nm (ΔX_{A1})+7.0 nm (ΔX_{GPIbα})+16.3 nm (43 linker residues X 3.8 Å/residue)−7.0 nm (ΔX A1-GPIbα).

FIGS. 5A and 5B show that at lower pulling rates (5 nm/s and 10 nm/s), most of the rupture events were observed in a narrow force range with a single Gaussian distribution.

FIG. 5C and D show that at higher pulling rates (20 nm/s and 40 nm/s), the rupture forces had a broader range of force and the range comprises two Gaussian-like distributions. The rupture force is the force required to break the interaction between the receptor-ligand pair

The Dudko-Szabo equation uses rupture force distribution during force rip experiments to estimate lifetime τ at constant force:

$$τ ( {F}_{0} + ( k - 1 / 2 ) Δ F ) = \frac{( {h}_{k} / 2 + {\sum }_{i = k + 1}^{N} {h}_{i} ) Δ F}{{h}_{k} F ( {F}_{0} + ( k - 1 / 2 ) Δ F )}$$

where τ(F) is bond lifetime at given force, F is the force loading rate, ΔF is the bin width that starts at F₀ and ends at F_{N}=F₀+NΔ, Fᵢ and Cᵢ is number of events in the ith bin, producing a height hi=Cᵢ/(N total ΔF) with Nₜₒₜₐₗ, total number of the events.

The inventors' single-molecule assay with optical tweezers demonstrates that tensile force on A1-GPIbα complex can induce flex-bond behavior. The bond exists in two states, each with distinct dissociation kinetics and response to applied force. At a force of 10 pN, the bond switched to the second, more force-resistant state. Activators of A1 binding to GPIbα, ristocetin and botrocetin, had distinct effects on flex-bond behavior. The assay developed here establishes a new method for single molecule measurements of reversible receptor-ligand bond interactions.

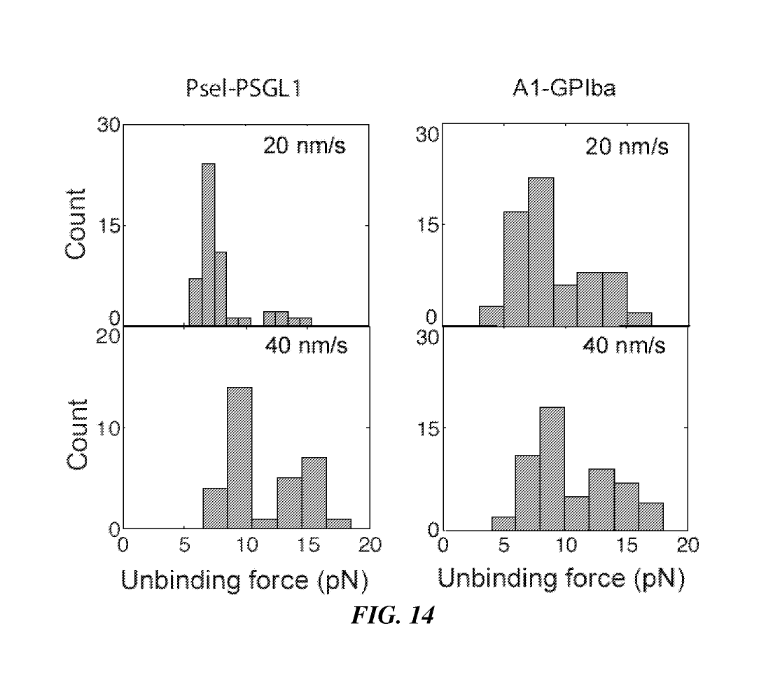

FIG. 14A and 14B show that at two pulling rates used (20 nm/s and 40 nm/s), the rupture forces for the P selectin-PSGL1 interaction and the VWF A1-GPIbα interaction had a broader range of force and the range comprises two Gaussian-like distributions although the flex-bond behavior of P selectin-PSGL1 is sufficiently matured at higher pulling rate (40 nm/s). The rupture force is the force required to break the interaction between the receptor-ligand pair.

### REFERENCES

X. Zhang, K. Halvorsen, C -Z Zhang, W. P. Wong, T. A. Springer, Science, 2009, 1330-1334.

O. K. Dudko, G. Hummer, and A. Szabo, PNAS, 2008, 15755-15760.

S. Miura, C. Q. Li, Z. Cao, H. Wang, M. R. Wardell, and J. E. Sadler, J B C, 2000, 7539-7546.

TABLE 1

| Antibody | Type | Target | Indication |
|---|---|---|---|
| Abciximab | chimeric | inhibition of glycoprotein | Cardiovascular disease |
| | | IIb/IIIa | |
| Adalimumab | human | inhibition of TNF-α signaling | Several auto-immune disorders |
| Alemtuzumab | humanized | CD52 | Chronic lymphocytic leukemia |
| Basiliximab | chimeric | IL-2Rα receptor (CD25) | Transplant rejection |
| Bevacizumab | humanized | Vascular endothelial growth | Colorectal cancer, Age related macular |
| | | factor (VEGF) | degeneration |
| Cetuximab | chimeric | epidermal growth factor | Colorectal cancer, Head and neck |
| | | receptor | cancer |
| Certolizumab | humanized | inhibition of TNF-α signaling | Crohn's disease |
| pegol | | | |
| Daclizumab | humanized | IL-2Rα receptor (CD25) | Transplant rejection |
| Eculizumab | humanized | Complement system protein | Paroxysmal nocturnal hemoglobinuria |
| | | C5 | |
| Efalizumab | humanized | CD11a | Psoriasis |
| Gemtuzumab | humanized | CD33 | Acute myelogenous leukemia (with |
| | | | calicheamicin) |
| Ibritumomab | murine | CD20 | Non-Hodgkin lymphoma (with |
| tiuxetan | | | yttrium-90 or indium-111) |
| Infliximab | chimeric | inhibition of TNF-α signaling | Several autoimmune disorders |
| Muromonab-CD3 | murine | T cell CD3 Receptor | Transplant rejection |
| Natalizumab | humanized | alpha-4 (α4) integrin, | Multiple sclerosis and Crohn's disease |
| Omalizumab | humanized | immunoglobulin E (IgE) | mainly allergy-related asthma |
| Palivizumab | humanized | an epitope of the RSV F | Respiratory Syncytial Virus |
| | | protein | |
| Panitumumab | human | epidermal growth factor | Colorectal cancer |
| | | receptor | |
| Ranibizumab | humanized | Vascular endothelial growth | Macular degeneration |
| | | factor A (VEGF-A) | |
| Rituximab | chimeric | CD20 | Non-Hodgkin lymphoma |
| Tositumomab | murine | CD20 | Non-Hodgkin lymphoma |
| Trastuzumab | humanized | ErbB2 | Breast cancer |

1. A method of screening for a modulator of an interaction between a receptor and a ligand pair, the method comprising:
a. contacting a ligand-bound-receptor protein with an agent;
b. extending the ligand-bound-receptor protein;
c. monitoring a signal that represents the protein existing in either a ligand-bound state or in a ligand-unbound state and the transition between the two states;
d. comparing the signal with a reference signal wherein a deviation from the reference indicate that the agent is a modulator.

2. The method of claim 1, wherein the reference is that of the ligand-bound-receptor protein in the absence of a modulator.

3. The method of claim 1, wherein the extending of the ligand-bound-receptor protein occurs with an optical tweezer or an atomic force microscope (AFM).

4. The method of claim 1, wherein the extending of the ligand-bound-receptor protein occurs with a mobile focus laser light, a cantilever, or a positioner in the AFM.

5. The method of claim 1, wherein the signal is a force required to dissociate the ligand receptor interaction and/or produce an increase in extension of the ligand-bound-receptor protein.

6. The method of claim 5, wherein a positive deviation of at least 10% from the reference indicates that the modulator is an agonist of the receptor-ligand interaction.

7. The method of claim 5, wherein a negative deviation of at least 10% from the reference indicates that the modulator is an antagonist of the receptor-ligand interaction.

8. The method of claim 1, wherein the signal is a rate of dissociation of the ligand receptor interaction and/or a dissociation constant of the rate.

9. The method of claim 8, wherein a negative deviation of at least 10% from the reference indicates that the modulator is an agonist of the receptor-ligand interaction.

10. The method of claim 8, wherein a positive deviation of at least 10% from the reference indicates that the modulator is an antagonist of the receptor-ligand interaction.

11. The method of claim 1, wherein the ligand-bound-receptor protein is a chimeric fusion protein comprising (1) a receptor or ligand-binding fragments thereof and (2) a ligand or receptor-binding fragment thereof, wherein the receptor or ligand-binding fragments thereof and the ligand or receptor-binding fragment thereof are fused together in a single polypeptide;

12. The method of claim 1, wherein the ligand-bound-receptor protein is a complex of two independent polypeptides wherein one polypeptide comprises a receptor or ligand-binding fragments thereof and the other polypeptide comprises a ligand or receptor-binding fragment thereof; wherein the complexing is by way of the ligand-receptor interaction; and wherein the two polypeptides are linked by non-covalent bonds located at non-ligand binding/non receptor-binding regions of the polypeptides.

13. The method of claim 1, wherein the ligand-bound-receptor protein is a complex of two independent polypeptides wherein one polypeptide comprises a receptor or ligand-binding fragments thereof and the other polypeptide comprises a ligand or receptor-binding fragment thereof; wherein the complexing is by way of the ligand-receptor interaction; and wherein the two polypeptides are linked by covalent bonds located at non-ligand binding/non receptor-binding regions of the polypeptides.

14. The method of claim 1, wherein the ligand is a natural ligand or an artificial ligand of the receptor.

15. (canceled)

16. The method of claim 1, wherein the receptor or ligand-binding fragments thereof and the ligand or receptor-binding fragment thereof are separated by a spacer linker peptide.

17. The method of claim 16, wherein the spacer linker peptide has at least one amino acid residue and up to about 200 amino acid residues.

18. The method of claim 1, wherein both amino and carboxyl ends of the protein are tethered to a handle for use with the optical tweezer or an AFM.

19. The method of claim 1, wherein only one end of the protein is tethered to a handle for use with the optical tweezers or atomic force microscope.

20. The method of claim 18, wherein the handle is a double-stranded DNA.

21. (canceled)

22. (canceled)

23. (canceled)

24. (canceled)

25. (canceled)

26. The method of claim 19, wherein the handle is a double-stranded DNA.
